(19) United States
(12) Patent Application Publication
(10) Pub. No.: US 2025/0339515 A1
(43) Pub. Date: Nov. 6, 2025

(54) METHODS FOR PREVENTING FOOD SENSITIZATION

(71) Applicant: MY-OR DIAGNOSTICS LTD., Zikhron Yaakov (IL)

(72) Inventors: Michael BRANDWEIN, Elazar (IL); Tamar LANDAU, Kiryat Ono (IL)

(21) Appl. No.: 18/880,161

(22) PCT Filed: Jul. 13, 2023
(86) PCT No.: PCT/IL2023/050738

§ 371 Date: Dec. 30, 2024

Related U.S. Application Data
(60) Provisional application No. 63/388,710, filed on Jul. 13, 2022.
(60) Provisional application No. 63/433,625, filed on Dec. 19, 2022.

(51) Int. Cl. A61K 39/35; A23L 33/00; A61K 9/00; A61K 39/00; A61P 37/08; C07K 14/415

(52) U.S. Cl. CPC A61K 39/35; A23L 33/40; A61K 9/0095; A61P 37/08; C07K 14/415; A61K 2039/542; A61K 2039/55; A61K 2039/577

(57) ABSTRACT

The present invention relates a method for preventing sensitization to a food allergen, such as including peanut protein and/or milk protein, in a subject in need thereof, including administering a composition including a low level of a food allergen, such as including a peanut protein and/or milk protein, to a mother breastfeeding the subject, thereby preventing sensitization to a food allergen, in the subject.

### CROSS-REFERENCE TO RELATED APPLICATIONS

This application claims the benefit of priority of U.S. Patent Application No. 63/338,710, titled “METHODS FOR PREVENTING FOOD SENSITIZATION”, filed 13 Jul. 2022, and of U.S. Patent Application No. 63/433,625, titled “METHOD FOR PREVENTING PEANUT PROTEIN SENSITIZATION”, filed 19 Dec. 2022. The contents of both applications are incorporated herein by reference in their entirety.

### FIELD OF INVENTION

The present invention, in some embodiments thereof, is in the field of treating or preventing food sensitization, such as, but not limited to peanut protein, or milk protein.

### BACKGROUND

The early introduction of allergenic foods, once thought to promote food allergies, has recently been endorsed by a number of professional organizations globally as a prevention strategy for food allergies. The exact timing of introduction, generally either between 4 and 6 months of age or within the first year of life, varies between these guidelines. Most allergens are consumed as solids as part of a healthy diet, thereby necessitating the prior developmental milestones that allow for solid food feeding sometime after 4 months of age. However, milk protein serves as an exception to the aforementioned rule, as it is often introduced in the form of infant formula soon after birth.

The study of milk formula introduction as a primary preventive measure for atopic conditions has been dominated by decades of industry-sponsored research purporting hydrolyzed formula's preventive abilities. The hypothesis driving these studies relied on the now-defunct paradigm of allergen avoidance as a food allergy avoidance measure. Many of the global guidelines point out the lack of evidence or lack of recommendation for using hydrolyzed formula for food allergy prevention but fail to address the growing body of work pointing towards whole milk protein formula's effects on milk allergy development.

Numerous studies examining the practice of whole milk protein infant formula feeding and subsequent allergic outcomes have emerged in the past years. Broadly speaking, their focus is either on brief infant formula supplementation in the immediate post-natal hospital or nursery stay or the extended supplementation in addition to or in lieu of breastfeeding. Both observational and interventional studies have suggested that the former increases the risk of cow's milk allergic outcomes, whereas the latter is protective against cow's milk allergic outcomes. However, recent consensus guidelines cite a lack of evidence, specifically randomized controlled trials, to support recommending infant formula supplementation as a milk allergy prevention modality.

There remains a need for methods for preventing sensitization to a food allergen, e.g., milk, or peanut protein, in a subject in need thereof, such as by maternal consumption of the allergen while breastfeeding.

### SUMMARY

According to a first aspect, there is provided a method for preventing sensitization to food allergen in a subject in need thereof, the method comprising administering a composition comprising an effective amount of the food allergen to any one of: (i) a mother being pregnant with the subject; (ii) a mother breastfeeding the subject, or both, thereby preventing sensitization to food allergen in the subject.

According to another aspect, there is provided a method for preventing sensitization to peanut protein in a subject in need thereof, the method comprising administering a composition comprising an effective amount of peanut protein to any one of: to any one of: (i) a mother being pregnant with the subject; (ii) a mother breastfeeding the subject, or both, thereby preventing sensitization to peanut protein in the subject.

In some embodiments, the food allergen is selected from the group consisting of: peanut, sesame, milk, egg, shellfish, tree nut, fruit, fish, wheat, soy, and any combination thereof.

In some embodiments, the effective amount comprises a weekly dosage of 2.5 to 7.5 gr.

In some embodiments, the composition is formulated for oral administration.

In some embodiments, the composition comprises the food allergen being encapsulated in a compound configured to inhibit or reduce degradation of the peanut protein in the gastrointestinal tract of the mother.

In some embodiments, preventing comprises reducing the severity of sensitization to the food allergen in the subject.

In some embodiments, preventing comprises reducing the titer of immunoglobulin E (IgE) targeting the food allergen in the subject, reducing wheal size developed by the subject in a skin prick test (SPT), or both.

In some embodiments, reducing comprises reducing the titer of IgE by at least 5% compared to a control subject.

In some embodiments, administering is multiple administering.

In some embodiments, multiple administering comprises daily administering.

In some embodiments, multiple administering comprises at least once a week administering.

In some embodiments, the composition is an edible composition comprising the food allergen.

In some embodiments, the subject is fed strictly on human breast milk.

In some embodiments, the method further comprises a step preceding the administering, comprising selecting a subject at a risk of developing sensitization to the food allergen.

In some embodiments, selecting is based on the subject having familial history of any one of: atopic dermatitis, food allergy, allergic rhinitis, asthma, eosinophilic esophagitis, or any combination thereof.

In some embodiments, selecting is further based on: parent of the subject planning to exclusively breastfeed the subject.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes vertical bar graphs showing the effects of cow milk (CM) introduction age on different measurements of allergy/sensitivity.

FIG. 2 include vertical bar graphs showing the outcomes of age of introduction on different measurements of allergy/sensitivity.

FIG. 3 includes a flow diagram of maternal peanut consumption groups in infants enrolled in the Learning Early About Peanut Allergy (LEAP) Study.

FIGS. 4A-4B include graphs showing that low maternal peanut consumption while breastfeeding protects against allergic endpoints at 60 months of age. Percent of population sensitized (4A) and allergic (4B) to peanut protein at 60 months of age by maternal consumption group.

FIGS. 5A-5B include graphs showing allergic sensitization over time in maternal peanut consumption groups. (5A) Percent of population sensitized at each scheduled clinical visit separated by maternal consumption group. The percentage of the population sensitized remains relatively stable in both the high and low consumption groups and increases gradually in the no maternal consumption group. (5B) Repeated SPT measurements from the same participants separated by maternal peanut consumption groups while breastfeeding.

FIGS. 6A-6C include graphs showing predictive ability of logistic regression model with lasso regularization for peanut sensitization at 60 months of age: Area under the receiver operating characteristic curves for the logistic regression model with lasso regularization (6A) and confusion matrix for the relevant parameters (6B). Precision recall curve for the model with associated area under the curve (6C).

FIG. 7 includes a graph showing maternal consumption of peanuts in the LEAP cohort.

FIGS. 8A-8B includes panels showing correlation matrix between external peanut exposure variables. (8A) Entire LEAP cohort; and (8B) avoidance group.

FIG. 9 includes a vertical bar graph showing different thresholds between low and high maternal weekly consumption of peanuts while breastfeeding, and the prevalence of infant peanut sensitivity (SPT≥3) at 60 months of age by groups.

FIG. 10 includes a horizontal bar graph showing comparison of thresholds in maternal peanut weekly consumption at breastfeeding. The threshold is the upper bound in group “Low” and the lower bound in group “High”. The X axis shows differences in percentage of the prevalence in SPT≥3 between the high and low consumption groups measured at 60 months. The Y axis show the different thresholds.

FIGS. 11A-11F include a flow chart, heat map, graphs, and a vertical bar graph. (11A) Overview of study design including cohort construction, characteristics of patients, and data splitting. (11B) Spearman correlation between predictor variables. (11C) Area under the receiver operating characteristic curve for random forest regression model with 95% CI's. (11D) Precision recall curve for the model is shown with the best point representing the maximum F1 score for the model. (11E) Relative feature importance of the features in the random forest regression model with standard deviation bars. (11F) Area under the receiver operating characteristic curves comparing the random forest regression algorithm developed in this study to the current best practice stratification techniques using either maternal history of food allergies or parents' history of atopic conditions.

FIG. 12 includes vertical bar graphs showing maternal systemic antibiotic prescriptions while pregnant in food allergy (FA) cohort and healthy cohort.

### DETAILED DESCRIPTION

According to one aspect, there is provided a method for preventing sensitization to a food allergen in a subject in need thereof.

In some embodiments, the method comprises administering a composition comprising an effective amount of a food allergen to a mother during pregnancy with the subject, a mother breastfeeding the subject, the subject, or any combination thereof, thereby preventing sensitization to food allergen in the subject.

In some embodiments, the method provides indirect prevention of sensitization to a food allergen in a subject in need thereof. In some embodiments, the method comprises indirectly preventing sensitization to a food allergen in the subject by administering a composition comprising an effective amount of a food allergen to a mother carrying the subject during pregnancy, breastfeeding the subject, or both.

In some embodiments, the term “indirectly” is to be meant that the composition comprising an effective amount of a food allergen is not provided to the subject per se.

In some embodiments, the term “indirectly” is to be meant that the composition comprising an effective amount of a food allergen is provided directly to a mother carrying the subject during pregnancy, breastfeeding the subject, or both, and from the mother to the subject.

In some embodiments, the food allergen comprises at least one protein of a food product, an edible composition, or both.

In some embodiments, a food allergen is selected from: peanut, sesame, milk, egg, shellfish, tree nut, fruit, fish, wheat, soy, any protein thereof, or any combination thereof.

In some embodiments, a food allergen comprises, consists of, suspected of comprising, or any combination thereof, a protein of a food product, e.g., a peanut protein, a milk protein, etc.

In some embodiments, the method comprises administering an effective amount of a composition comprising the food allergen.

In some embodiments, the method comprises administering a therapeutically effective amount of a composition comprising the food allergen.

In some embodiments, the method comprises administering low level of the food allergen to a subject, a mother breastfeeding the subject, or both.

In some embodiments, the method further comprises administering a food allergen, or composition comprising same, to mother during pregnancy or while being pregnant. In some embodiments, the method further comprises administering a food allergen, or composition comprising same, to mother during pregnancy or while being pregnant with the subject.

In some embodiments, the further comprises administering the food allergen, or composition comprising same, to a mother carrying the subject while being a fetus (e.g., during pregnancy).

According to some embodiments, there is provided a method for preventing sensitization to peanut protein in a subject in need thereof, the method comprising administering a composition comprising peanut protein to a subject, a mother breastfeeding the subject, or both, thereby preventing sensitization to peanut protein in the subject.

In some embodiments, the method comprises administering an effective amount of a composition comprising peanut protein.

In some embodiments, the method comprises administering a therapeutically effective amount of a composition comprising peanut protein.

In some embodiments, the method comprises administering low level of peanut protein to a mother breastfeeding the subject.

In some embodiments, administering comprises administering a weekly dose of peanut protein.

In some embodiments, a weekly dose of peanut protein, such as included in a composition as disclosed herein, is in an amount of not more than 2 g, 2.5 g, 3 g, 3.5 g, 4 g, 4.5, 5 g, 5.5 g, 6 g, 6.5 g, 7 g, 7.5 g, 8 g, 10 g, or any value and range therebetween, per week. Each possibility represents a separate embodiment of the invention.

In some embodiments, a weekly dose of peanut protein, such as included in a composition as disclosed herein, is in an amount ranging from 2.5 g to 7.5 gr, 2 g to 10 gr, 2 g to 9.5 gr, 2.5 g to 9 gr, 2 g to 8.5 gr, 3.5 g to 9.5 gr, 2 g to 5 gr, 4 g to 7.5 gr, 3.5 g to 7 gr, or 3 g to 7, per week. Each possibility represents a separate embodiment of the invention.

In some embodiments, the method further comprises administering a peanut protein, or composition comprising same, to mother during pregnancy or while being pregnant with the subject.

In some embodiments, the further comprises administering the peanut protein, or composition comprising same, to a mother carrying the subject while being a fetus (e.g., during pregnancy).

In some embodiments, the composition is formulated for oral administration.

In some embodiments, the composition is an oral composition.

In some embodiments, the composition is a nutraceutical composition.

In some embodiments, the composition is a pharmaceutical composition.

In some embodiments, the composition is an edible composition.

In some embodiments, the composition further comprises an acceptable carrier, excipient, or diluent.

In some embodiments, a carrier comprises nutraceutical carrier, pharmaceutical carrier, or both.

In some embodiments, the carrier is suitable for oral administration of the composition disclosed herein.

In some embodiments, the composition comprises the food allergen being encapsulated in a compound configured to inhibit or reduce degradation of the food allergen in the gastrointestinal tract of the breastfeeding, pregnant, or both, mother.

In some embodiments, the composition comprises the peanut protein being encapsulated in a compound configured to inhibit or reduce degradation of the peanut protein in the gastrointestinal tract of the breastfeeding, pregnant, or both, mother.

In some embodiments, the compound comprises or is plastic.

In some embodiments, the present invention is directed to a composition comprising peanut protein derived from an extract of a plant or a part thereof. In some embodiments, an extract of plant or a part thereof is derived from a plant comprising a peanut protein. In some embodiments, the extract as disclosed herein is derived from a peanut plant (Arachis hypogaea).

In some embodiments, a plant part comprises any part derived from a peanut plant, e.g., seed, leaf, flower, root, etc.

In some embodiments, a plant part comprises or is a seed of a peanut plant.

In some embodiments, a peanut plant may be known as groundnut, goober, pindar or monkey nut.

In some embodiments, the extract as disclosed herein is derived from a specific species or cultivar of the peanut plant (Arachis hypogaea) or a part thereof.

In some embodiments, the peanut protein is present as a highly purified extract of peanut plant (Arachis hypogaea) or a part thereof.

In some embodiments, the peanut protein is a synthetically or biosynthetically produced peanut protein, e.g., recombinantly expressed peanut protein.

As used herein, the term “synthetically produced” refers to the peanut protein being produced by or synthesized by man. In some embodiments, synthetic comprises “man-made”. In some embodiments, synthetic comprises cultured, grown, processed, manipulated, isolated, extracted, purified, or any combination thereof, in-vitro.

In some embodiments, preventing comprises reducing the severity of sensitization to the food allergen in the subject. In some embodiments, preventing comprises reducing the severity of sensitization to the peanut protein in the subject.

In some embodiments, preventing comprises reducing the severity, rate, or both, of allergic reaction of the subject to a food allergen. In some embodiments, preventing comprises reducing the severity, rate, or both, of allergic reaction of the subject to peanut protein.

In some embodiments, preventing comprises reducing the titer of immunoglobulin E (IgE) targeting a food allergen, reducing wheal size developed by the subject in a skin prick test (SPR), or both, in the subject. In some embodiments, preventing comprises reducing the titer of IgE targeting peanut protein, reducing wheal size developed by the subject in a skin prick test (SPR), or both, in the subject. In some embodiments, preventing comprises reducing the titer of IgE targeting milk protein in the subject, reducing wheal size developed by the subject in a skin prick test (SPT), or both.

In some embodiments, reducing comprises reducing the titer of IgE by at least 5% compared to a control subject.

In some embodiments, a control subject comprises a subject not being treated according to the method disclosed herein. In some embodiments, a control subject is not administered with the composition according to the method of the invention. In some embodiments, a control subject comprises a subject not afflicted with sensitization or allergy to a food allergen. In some embodiments, a control subject comprises a subject not afflicted with sensitization or allergy to peanut protein. In some embodiments, a control subject comprises a subject having non or low predisposition to developing sensitization or allergy to a food allergen. In some embodiments, a control subject comprises a subject having non or low predisposition to developing sensitization or allergy to peanut protein. In some embodiments, a control subject comprises a subject having low risk to developing sensitization or allergy to a food allergen. In some embodiments, a control subject comprises a subject having low risk to developing sensitization or allergy to peanut protein.

In some embodiments, administering comprises multiple administering.

In some embodiments, multiple administering comprises daily administering.

In some embodiments, multiple administering comprises at least once a week administering.

In some embodiments, multiple administering comprises at least once a week, at least twice a week, at least once every other week, at least once in two weeks, at least once in three weeks, at least once a month administering, or any value and range therebetween. Each possibility represents a separate embodiment of the invention. In some embodiments, multiple administering comprises once a week to once a month administering.

In some embodiments, the subject is fed on a diet consisting of human breast milk. In some embodiments, the subject is being breast fed. In some embodiments, the subject is fed on pumped human breast milk. In some embodiments, the subject is fed strictly on human breast milk. In some embodiments, the subject is exclusively breastfed. In some embodiments, the subject is fed strictly on human breast milk during the period of which the breastfeeding mother is administered with the composition as disclosed herein.

In some embodiments, the composition is administered in a therapeutically effective amount. In some embodiments, a therapeutically effective amount relates to the subject.

In some embodiments, the composition is administered in an effective amount. In some embodiments, an effective amount relates to the mother breastfeeding the subject.

In some embodiments, an effective amount comprises the amount of a food allergen, such as administered using the composition disclosed herein, which is delivered to a breastfeeding mother so as to elicit the effect as disclosed herein, e.g., preventing or reducing sensitization, allergic reaction, or both, to a food allergen in a subject, e.g., being breastfed. In some embodiments, an effective amount comprises the amount of a food allergen, such as administered using the composition disclosed herein, which is delivered to a breastfeeding mother so as to elicit the effect as disclosed herein, e.g., preventing or reducing sensitization, allergic reaction, or both, to a food allergen in a subject, e.g., being breastfed.

In some embodiments, peanut protein comprises any edible composition, food, foodstuff, or any equivalent thereof, or any combination thereof, that induces an immune response when orally consumed by a subject. In some embodiments, peanut protein comprises or is a protein or a plurality of proteins derived from an edible composition, or food product, comprising peanuts. In some embodiments, an immune response comprises production of an immunoglobulin in a response to the consumption (e.g., oral consumption), contact (e.g., with the skin), or both, with peanut protein. In some embodiments, the immunoglobulin is or comprises IgE.

In one embodiment, a subject as described herein is an infant. In another embodiment, the subject is a child. In one embodiment, the infant is 2 weeks old at most. In another embodiment, the infant is approximately 3 months old.

It is to be stressed out here, that sensitization alone is not sufficient to diagnose a food allergy, e.g., peanut, milk, etc., nor is synonymous or equivalent thereto.

In some embodiments, sensitization and allergy are not synonymous or equivalent.

In some embodiments, sensitization is characterized by: the development of specific IgE (sIgE) capable of targeting and/or binding to a food allergen (or a portion thereof) by a subject, lack of clinical symptoms after the subject has been exposed to a food allergen, or both. In some embodiments, sensitization is characterized by: the development of specific IgE (sIgE) capable of targeting and/or binding to peanut protein by a subject, lack of clinical symptoms after the subject has been exposed to peanut protein, or both.

In some embodiments, allergy to a food allergen is characterized by the development of clinical symptoms after a subject has been exposed to the food allergen. In some embodiments, allergy such as to peanut protein, milk protein, etc. is characterized by the development of clinical symptoms after a subject has been exposed to the protein, e.g., peanut, milk, etc..

Methods for determining IgE titer, clinical symptoms, or both, are common and would be apparent to one of ordinary skill in the art of medicine.

In some embodiments, a subject in need of prevention according to the herein disclosed method is at risk of developing sensitization to a food allergen. In some embodiments, the method disclosed herein is directed to prevent sensitization to a food allergen. In some embodiments, a subject in need of prevention according to the herein disclosed method is at risk of developing sensitization to food derived protein, such as peanut protein, milk protein, etc. In some embodiments, the method disclosed herein is directed to prevent sensitization to peanut protein. In some embodiments, the method disclosed herein is directed to prevent sensitization to milk protein. In some embodiments, the method disclosed herein is directed to prevent sensitization to a food derived protein.

In some embodiments, preventing comprises reducing the severity or tendency of the subject to developing (food) allergy to the food allergen. In some embodiments, preventing comprises reducing the severity or tendency of the subject to developing (food) allergy to a food derived protein. In some embodiments, preventing comprises reducing the severity or tendency of the subject to developing (food) allergy to a peanut protein. In some embodiments, preventing comprises reducing the severity or tendency of the subject to developing (food) allergy to a milk protein.

In some embodiments, the method comprises reducing a wheal size to 0.1 mm at most, 0.5 mm at most, 1.0 mm at most, 2.5 mm at most, 2 mm at most, or any value and range therebetween. Each possibility represents a separate embodiment of the invention. In some embodiments, the method comprises reducing a wheal size to 0.1-2.0 mm, 0.2-1.9 mm, 0.3-1.95 mm, or 0.2-2.0 mm. Each possibility represents a separate embodiment of the invention.

In some embodiments, reducing is by at least 5%, at least 15%, at least 25%, at least 50%, at least 100%, at least 200%, at least 350%, at least 500%, at least 750%, at least 1,000%, or any value and range therebetween. Each possibility represents a separate embodiment of the invention. In some embodiments, reducing is by 5-200%, 5-50%, 100-250%, 200-750%, 10-350%, 90-450%, or 150-1,000%. Each possibility represents a separate embodiment of the invention. In some embodiments, reducing is compared to a control subject, as described herein.

As used herein, the term “prevention” refers to reducing susceptibility, delay, prevention, suppression, or inhibition of the onset of a disease, disorder, or condition. As used in accordance with the presently described subject matter, the term “prevention” relates to a process of prophylaxis in which a subject is exposed to the presently described compositions or composition prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

In some embodiments, preventing comprises reducing the susceptibility of a subject administered with the composition according to the method of the invention, to develop oral sensitization, food allergy, or both, to a food allergen. In some embodiments, a subject administered with the composition according to the method of the invention, has reduced susceptibility of developing oral sensitization, food allergy, or both, to a food allergen, compared to a control subject.

In some embodiments, preventing comprises reducing the susceptibility of a subject administered with the composition according to the method of the invention, to develop oral sensitization, food allergy, or both, to a protein derived from food, comprising a peanut protein, a milk protein, or both. In some embodiments, a subject administered with the composition according to the method of the invention, has reduced susceptibility of developing oral sensitization, food allergy, or both, to a protein derived from a food product, comprising: peanut protein, milk protein, or both, compared to a control subject.

As used herein, the term “at risk” refers to the susceptibility of a subject to developing a condition, such as sensitization to peanut protein. In some embodiments, detecting or determining a risk comprises detecting the presence of the disease itself. In some embodiments, detecting or determining a risk comprises any one of: determining the susceptibility of the subject to developing the condition, having a poor prognosis for the condition, having increased severity of the condition, or any combination thereof.

In some embodiments, “at risk” refers to increased probability of developing, or having increased predisposition, compared to a control. In some embodiments, the control comprises the average or common population.

The terms “infant”, “baby”, “preemie”, “newborn”, are used herein interchangeably.

In some embodiments, preventing a sensitization to a food allergen, food allergy, or both (e.g., “the condition”) comprises reducing the condition severity, delaying the condition onset, reducing the condition cumulative incidence, or any combination thereof.

In some embodiments, the composition further comprises an additional biomedical active agent.

In some embodiments, the additional biomedical active agent is selected: a mineral, a vitamin, probiotics, or any combination thereof.

Effective doses of the compositions as disclosed herein, for treatment of conditions or diseases vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages may be titrated using routine methods known to those of skill in the art to optimize safety and efficacy. The pharmaceutical compositions of the invention thus may include a “therapeutically effective amount.” A “therapeutically effective amount” refers to an amount effective, at dosages and for periods of time necessary, to achieve the desired therapeutic result. A therapeutically effective amount of a molecule may vary according to factors such as the disease state, age, sex, and weight of the individual, and the ability of the molecule to elicit a desired response in the individual. A therapeutically effective amount is also one in which any toxic or detrimental effects of the molecule are outweighed by the therapeutically beneficial effects.

Furthermore, a skilled artisan would appreciate that the term “therapeutically effective amount” may encompass total amount of each active component of the pharmaceutical composition or method that is sufficient to show a meaningful patient benefit, i.e., treatment, healing, prevention or amelioration of the relevant medical condition, or an increase in rate of treatment, healing, prevention or amelioration of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

The amount of a compound that will be effective in the treatment of a particular disorder or condition, also will depend on the nature of the disorder or condition and can be determined by standard clinical techniques.

For therapeutic purposes, the active compounds of this invention are ordinarily combined with one or more adjuvants appropriate to the indicated route of administration.

Compositions as disclosed herein, to be used according to the method of the invention, in some embodiments, optionally comprise an additional agent selected from any pharmaceutically or nutraceutically acceptable carrier, adjuvant, and vehicle.

According to another aspect, there is provided a method for preventing sensitization to milk protein, in a subject in need thereof.

According to another aspect, there is provided a method for preventing sensitization to milk protein in a subject in need thereof, comprising administering to the subject a therapeutically effective amount of a composition comprising A2 milk. In some embodiments, the administering commences when the subject being 3 months old at most.

In some embodiments, the method comprises: (a) selecting a subject at a risk of developing sensitization to milk protein; and (b) orally administering to the subject a therapeutically effective amount of a composition comprising the milk protein in a non-hydrolyzed form, thereby preventing sensitization to milk protein in the subject.

In some embodiments, the method comprises administering to the subject a therapeutically effective amount of a composition comprising A2 milk.

In some embodiments, non-hydrolyzed milk protein comprises whole milk protein, intact milk protein, or both.

In some embodiments, the composition comprises cow milk, goat milk, sheep milk, yak milk, buffalo milk, camel milk, or any combination thereof. In some embodiments, the composition comprises cow's milk. In some embodiments, the composition comprises A2 milk.

The term “A2 milk” is common and would be apparent to one of ordinary skill in the art, and generally refers to milk that is essentially devoid of A1 β casein protein (term “A1 milk”).

In some embodiments, milk protein comprises any edible composition, food, foodstuff, or any equivalent thereof, or any combination thereof, that induces an abnormal immune response when orally consumed by a subject. In some embodiments, milk protein comprises or is a protein or a plurality of proteins derived from an edible composition, or food product, such as, but not limited to a dairy product. In some embodiments, an abnormal immune response comprises production of an immunoglobulin in a response to the consumption (e.g., oral consumption), contact (e.g., with the skin), or both, with milk protein. In some embodiments, the immunoglobulin is or comprises IgE.

In some embodiments, the administering commences when the subject is at least 1 day old, at least 2 days old, at least 3 days old, at least 4 days old, at least 5 days old, at least 7 days old, at least 9 days old, at least 10 days old, at least 12 days old, at least 14 days old, at least 16 days old, at least 19 days old, at least 21 days old, at least 25 days old, at least 28 days old, or at least 30 days old, or any value ad range therebetween. Each possibility represents a separate embodiment of the invention.

In some embodiments, the administering commences when the subject is 1 day old at most, 2 days old at most, 3 days old at most, 4 days old at most, 5 days old at most, 7 days old at most, 9 days old at most, 10 days old at most, 12 days old at most, 14 days old at most, 16 days old at most, 19 days old at most, 21 days old at most, 25 days old at most, 28 days old at most, or 30 days old at most, 1 month at most, 2 months at most, 3 months at most, 4 months at most, 5 months at most, 6 months at most, 8 months at most, or any value ad range therebetween. Each possibility represents a separate embodiment of the invention. In some embodiments, the administering commences when the subject is 1 day to 14 days, 2 days to 30 days, 1 day to 10 days old, 4 days to 16 days old, 5 days to 15 days old, 1 day to 7 days, or 3 days to 18 days, 1 week to 5 weeks, 1 month to 6 months, 2 month to 8 months, 3 month to 9 months, or 1 month to 8 months. Each possibility represents a separate embodiment of the invention. In some embodiments, the administering commences when the subject is 3 months old at most.

In some embodiments, sensitization and allergy are not synonymous or equivalent.

In some embodiments, sensitization is characterized by: the development of specific IgE (sIgE) capable of targeting and/or binding to milk protein by a subject, lack of clinical symptoms after the subject has been exposed to milk protein, or both.

In some embodiments, allergy to milk protein is characterized by the development of clinical symptoms after a subject has been exposed to the milk protein.

Methods for determining IgE titer, clinical symptoms, or both, are common and would be apparent to one of ordinary skill in the art of medicine.

In some embodiments, a subject in need of prevention according to the herein disclosed method is at risk of developing sensitization to milk protein. In some embodiments, the method disclosed herein is directed to prevent sensitization to milk protein.

In some embodiments, preventing comprises reducing the severity or tendency of the subject to developing (food) allergy to the milk protein.

In some embodiments, preventing comprises reducing the severity of sensitization to the milk protein in the subject.

In some embodiments, the method comprises reducing a wheal size to 0.1 mm at most, 0.5 mm at most, 1.0 mm at most, 2.5 mm at most, 2 mm at most, or any value and range therebetween. Each possibility represents a separate embodiment of the invention.

In some embodiments, the method comprises reducing a wheal size to 0.1-2.0 mm, 0.2-1.9 mm, 0.3-1.95 mm, or 0.2-2.0 mm. Each possibility represents a separate embodiment of the invention.

In some embodiments, reducing is by at least 5%, at least 15%, at least 25%, at least 50%, at least 100%, at least 200%, at least 350%, at least 500%, at least 750%, at least 1,000%, or any value and range therebetween. Each possibility represents a separate embodiment of the invention. In some embodiments, reducing is by 5-200%, 5-50%, 100-250%, 200-750%, 10-350%, 90-450%, or 150-1,000%. Each possibility represents a separate embodiment of the invention. In some embodiments, reducing is compared to a control subject, as described herein.

In reducing comprises reducing the titer of IgE by at least 5% compared to a control subject.

As used herein, the term “prevention” refers to reducing the susceptibility, delay, prevention, suppression, or inhibition of the onset of a disease, disorder, or condition. As used in accordance with the presently described subject matter, the term “prevention” relates to a process of prophylaxis in which a subject is exposed to the presently described compositions or composition prior to the induction or onset of the disease/disorder process. This could be done where an individual has a genetic pedigree indicating a predisposition toward occurrence of the disease/disorder to be prevented. For example, this might be true of an individual whose ancestors show a predisposition toward certain types of, for example, inflammatory disorders. The term “suppression” is used to describe a condition wherein the disease/disorder process has already begun but obvious symptoms of the condition have yet to be realized. Thus, the cells of an individual may have the disease/disorder, but no outside signs of the disease/disorder have yet been clinically recognized. In either case, the term prophylaxis can be applied to encompass both prevention and suppression. Conversely, the term “treatment” refers to the clinical application of active agents to combat an already existing condition whose clinical presentation has already been realized in a patient.

In some embodiments, preventing comprises reducing the susceptibility of a subject administered with the composition according to the method of the invention, to develop oral sensitization, food allergy, or both, to milk protein. In some embodiments, a subject administered with the composition according to the method of the invention, has reduced susceptibility of developing oral sensitization, food allergy, or both, to milk protein, compared to a control subject. In some embodiments, a control subject is not administered with the composition according to the method of the invention.

As used herein, the term “at risk” refers to the susceptibility of a subject to developing a condition, such as sensitization to milk protein. In some embodiments, detecting or determining a risk comprises detecting the presence of the disease itself. In some embodiments, detecting or determining a risk comprises any one of: determining the susceptibility of the subject to developing the condition, having a poor prognosis for the condition, having increased severity of the condition, or any combination thereof.

In some embodiments, “at risk” refers to increased probability of developing, or having increased predisposition, compared to a control. In some embodiments, the control comprises the average population.

The terms “infant”, “baby”, “preemie”, “newborn”, are used herein interchangeably.

In some embodiments, preventing a sensitization to a food allergen, food allergy, or both (e.g., “the condition”) comprises reducing the condition severity, delaying the condition onset, reducing the condition cumulative incidence, or any combination thereof.

In some embodiments, administering is multiple administering.

In some embodiments, the method comprises multiple administering. In some embodiments, the method comprises daily administering. In some embodiments, multiple administering comprises daily administering. In some embodiments, multiple administering comprises at least once a week, at least twice a week, at least once every other week, at least once in two weeks, at least once in three weeks, at least once a month administering, or any value and range therebetween. Each possibility represents a separate embodiment of the invention. In some embodiments, multiple administering comprises once a week to once a month administering.

In some embodiments, the composition is a pharmaceutical composition or a nutraceutical composition.

In some embodiments, the composition is an edible composition.

In some embodiments, the composition further comprises an additional biomedical active agent.

In some embodiments, the additional biomedical active agent is selected: a mineral, a vitamin, probiotics, or any combination thereof.

In some embodiments, the mineral comprises iron.

In some embodiments, the vitamin comprises vitamin D, vitamin B12, or any combination thereof.

In some embodiments, the composition is formulated in any way known to one of skill in the art as suitable for administration in a baby, an infant, or a newborn.

In some embodiments, the composition is administered in conjugation to breast feeding.

In some embodiments, the subject is fed strictly on human breast milk. In some embodiments, the subject is exclusively breastfed.

In some embodiments, the composition comprises cow milk formula (CMF).

As used herein, the term “CMF” refers to any human breast milk replacement produced artificially and/or synthetically by man. Types of CMF are common and would be apparent to one of ordinary skill in the art.

According to some embodiments, there is provided a method for preventing sensitization or food allergy to a non-human milk protein (hMP) in a subject in need thereof, wherein the subject is an infant strictly or exclusively fed on human breast milk.

In some embodiments, the herein disclosed method is directed to providing oral tolerance of a subject to a non-hMP. In some embodiments, the herein disclosed method and/or composition are directed to provide oral tolerance of a subject to a non-hMP and not as a replacement to human milk protein and/or breast feeding. In some embodiments, replacement comprises partial or full replacement.

In some embodiments, the subject is fed on a diet consisting of human breast milk. In some embodiments, the subject is being breast fed. In some embodiments, the subject is fed on pumped human breast milk.

In some embodiments, selecting is based on the subject having familial history of: atopic dermatitis, food allergy, allergic rhinitis, asthma, eosinophilic esophagitis, or any combination thereof, or any combination thereof.

In some embodiments, the selecting is further based on a parent of the subject planning to exclusively breastfeed the subject.

In some embodiments, selecting is based on the subject being characterized by having an above average/over a predetermined threshold of transepidermal water loss (TEWL) value.

Methods for determining TEWL are common and would be apparent to one of skill in the art, as well as what is considered normal, proper, adequate, or average, TEWL value.

According to another aspect, there is provided a composition comprising an effective amount of a food allergen for use in prevention of sensitization to a food allergen in a subject in need thereof, wherein the composition being administered to any one of: (i) a mother being pregnant with the subject; (ii) a mother breastfeeding the subject, or both.

According to another aspect, there is provided a composition comprising an effective amount of a peanut protein for use in prevention of sensitization to peanut protein in a subject in need thereof, wherein the composition being administered to any one of: (i) a mother being pregnant with the subject; (ii) a mother breastfeeding the subject, or both.

In some embodiments, the composition is for use according to the method of the invention.

In some embodiments, the composition is a pharmaceutical composition, or a nutraceutical composition.

In some embodiments, the composition further comprises a pharmaceutical carrier, or a nutraceutical carrier.

As used herein, the term “carrier”, “excipient”, or “adjuvant” refers to any component of a composition, that is not the active agent, such as, but not limited to the bacterial consortium disclosed herein.

In some embodiments, the carrier is a physiologically acceptable carrier. In one embodiment, the phrases “physiologically acceptable carrier” and “pharmaceutically acceptable carrier” which be interchangeably used refer to a carrier or a diluent that does not cause significant irritation to an organism and does not abrogate the biological activity and properties of the administered compound. An adjuvant is included under these phrases. In one embodiment, “excipient” refers to an inert substance added to a pharmaceutical composition to further facilitate administration of an active ingredient. In one embodiment, excipients include calcium carbonate, calcium phosphate, various sugars and types of starch, cellulose derivatives, gelatin, vegetable oils and polyethylene glycols.

As used herein, the term “carrier”, “excipient”, or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

According to another aspect, there is provided a method of determining or predicting predisposition to developing sensitization to a food allergen in a subject.

According to another aspect, there is provided a method of generating a classifier suitable for predicting predisposition of a subject to a food allergen, by at least one processor.

In some embodiments, the method comprises: (a) receiving presence and/or level of a plurality of factors listed in Table 14 in a population of subjects afflicted with sensitization to the food allergen (‘sensitized population’) and in a population of subjects not afflicted with sensitization to the food allergen (non-sensitized population); and (b) at a training stage, training a machine learning model on a training set comprising: (i) presence and/or level of the plurality of factors is subjects from the populations (sensitized and desensitized or non-sensitized), and (ii) labels associated with the sensitization and/or desensitization of a subject in the populations to the food allergen; thereby generating a classifier suitable for predicting predisposition of a subject to the food allergen.

In some embodiments, the method comprises: (a) receiving presence and/or level of a plurality of factors listed in Table 14

i. in a population of subjects afflicted with sensitization to a food allergen ('sensitized population'); and

ii. in the subject;

(b) calculate for factors of the plurality of factors a predisposition score, wherein the predisposition score is based on the similarity of the factor presence and/or level in the subject to the factor presence and/or level in the sensitized population;

(c) classify a factor of the plurality of factors with a predisposition score beyond a predetermined threshold as a predisposition-associated factor; and

(d) applying a trained machine learning algorithm to the predisposition-associated factor, wherein the trained machine learning algorithm outputs a predisposition score,

thereby predicting predisposition of the subject to a food allergen.

In some embodiments, the method comprises:

(a) receiving presence and/or level of a plurality of factors listed in Table 14 in a population of subjects afflicted with sensitization to a food allergen ('sensitized population'); and

(b) at a training stage, training a machine learning model on a training set comprising:

i. the plurality of factors, and

ii. labels associated with an outcome in the population of subjects afflicted with sensitization to a food allergen (‘sensitized population’);

thereby generating a classifier suitable for predicting predisposition of the subject to a food allergen.

In some embodiments, the method further comprises at an inference stage, applying the classifier to a target set of plurality of factors associated with a target subject, thereby predicting predisposition of the subject to a food allergen.

The method further comprises a step comprising calculating from factors of the plurality of factors a predisposition score. In some embodiments, the calculating step is performed after step (a) and before step (b) of the method.

TABLE 14
Plurality of factors

| | Presence and/or | |
|---|---|---|
| Factor | Level | Description |
| Gender | Male/Female | Gender |
| Season of Birth | Autumn/Spring/ | Season of Birth |
| | Summer/Winter | |
| Firstborn | Yes/No | Firstborn |
| Percent of | Fractional in | Number of siblings with an |
| Siblings with an | range 0-100 | atopic condition divided by the |
| Atopic | | total number of siblings |
| Condition | | |
| Siblings with | Integer in range 0-5 | Number of siblings with a food |
| Food Allergy | | allergy |
| Parental Atopic | Integer in range 0-8 | Total number of atopic |
| History | | conditions diagnosed in either |
| | | parent's medical file |
| Systemic | Integer in range 0-3 | Number of prescriptions for |
| Antibiotics | | systemic antibiotics during the |
| While Pregnant | | pregnancy |
| Active Atopy | Integer in range 0-3 | Number of atopic diagnoses |
| While Pregnant | | during the pregnancy |
| Infant Atopic | Yes/No | Diagnosis of atopic dermatitis |
| Dermatitis | | before a food allergy diagnosis, |
| | | but no later than 4 months of age |

In some embodiments, a subject with a number of predisposition-associated factors above a predetermined number is predicted to be sensitized or developing sensitization to a food allergen.

In some embodiments, a subject with a number of predisposition-associated factors at or below a predetermined number is predicted not to be sensitized or developing sensitization to a food allergen.

According to another aspect, there is provided a system comprising: at least one hardware processor; and a non-transitory computer-readable storage medium having stored thereon program instructions, the program instructions executable by the at least one hardware processor to: receive presence and/or level for each of a plurality of factors listed in Table 14 in a population of subjects afflicted with sensitization to the food allergen (‘sensitized population’) and in a population of subjects not afflicted with sensitization to the food allergen (non-sensitized population); calculate for factors of the plurality of factors a predisposition score, wherein the predisposition score is based on the similarity of the factor presence and/or level in the subject to the factor presence and/or level in the sensitized population; and at a training stage, train a machine learning model on a training set comprising: (i) the calculated predisposition score, and (ii) labels associated with an outcome of the predisposition score in each of the subjects, to generate a classifier suitable for predicting predisposition in a target subject to sensitization to a food allergen.

By another aspect there is provided, a computer program product comprising a non-transitory computer-readable storage medium having program code or instructions embodied thereon or therewith, the program code executable by at least one hardware processor to perform a method of the invention.

In some embodiments, there is provided a computer program product comprising a non-transitory computer-readable storage medium having program instructions embodied therewith, the program instructions executable by at least one hardware processor to: receive, presence and/or level for each of a plurality of factors listed in Table 14 in a population of subjects afflicted with sensitization to the food allergen (‘sensitized population’) and in a population of subjects not afflicted with sensitization to the food allergen (non-sensitized population), calculate for factors of the plurality of factors a predisposition score, wherein the predisposition score is based on the similarity of the factor presence and/or level in the subject to the factor presence and/or level in the sensitized population, and at a training stage, train a machine learning model on a training set comprising: (i) the calculated predisposition score, and (ii) labels associated with an outcome of the predisposition score in each of the subjects, to generate a classifier suitable for predicting predisposition in a target subject to sensitization to a food allergen.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire. Rather, the computer readable storage medium is a non-transient (i.e., not-volatile) medium.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

These computer readable program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

### General

In the discussion unless otherwise stated, adjectives such as “substantially” and “about” modifying a condition or relationship characteristic of a feature or features of an embodiment of the invention, are understood to mean that the condition or characteristic is defined to within tolerances that are acceptable for operation of the embodiment for an application for which it is intended. Unless otherwise indicated, the word “or” in the specification and claims is considered to be the inclusive “or” rather than the exclusive or, and indicates at least one of, or any combination of items it conjoins.

It should be understood that the terms “a” and “an” as used above and elsewhere herein refer to “one or more” of the enumerated components. It will be clear to one of ordinary skill in the art that the use of the singular includes the plural unless specifically stated otherwise. Therefore, the terms “a”, “an”, and “at least one” are used interchangeably in this application.

For purposes of better understanding the present teachings and in no way limiting the scope of the teachings, unless otherwise indicated, all numbers expressing quantities, percentages or proportions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained. At the very least, each numerical parameter should at least be construed in light of the number of reported significant digits and by applying ordinary rounding techniques.

In the description and claims of the present application, each of the verbs, “comprise,” “include” and “have” and conjugates thereof, are used to indicate that the object or objects of the verb are not necessarily a complete listing of components, elements or parts of the subject or subjects of the verb.

Other terms as used herein are meant to be defined by their well-known meanings in the art.

Unless specifically stated or obvious from context, as used herein, the term “or” is understood to be inclusive.

Throughout this specification and claims, the word “comprise,” or variations such as “comprises” or “comprising,” indicate the inclusion of any recited integer or group of integers but not the exclusion of any other integer or group of integers.

As used herein, the term “consists essentially of,” or variations such as “consist essentially of” or “consisting essentially of,” as used throughout the specification and claims, indicate the inclusion of any recited integer or group of integers, and the optional inclusion of any recited integer or group of integers that do not materially change the basic or novel properties of the specified method, structure or composition.

As used herein, the terms “comprises”, “comprising”, “containing”, “having” and the like can mean “includes”, “including”, and the like; “consisting essentially of or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments. In one embodiment, the terms “comprises,” “comprising, “having” are/is interchangeable with “consisting”.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Various embodiments and aspects of the present invention as delineated hereinabove and as claimed in the claims section below find experimental support in the following examples. Reference is now made to the following examples, which together with the above descriptions illustrate some embodiments of the invention in a non-limiting fashion.

### Materials and methods

### Participants

The inventors performed a secondary analysis of infant data from a LEAP study, a randomized, open-label, controlled trial designed to determine whether early consumption of peanut in high-risk infants would reduce the prevalence of peanut allergy at 5 years of age. This study was not part of the original LEAP study statistical analysis plan or protocol.

The complete description of the LEAP study design and study procedures have been described previously. Briefly, infants with severe eczema and/or egg allergy were enrolled between 4 and 11 months of age and randomly assigned to either peanut avoidance or peanut consumption groups. Infants in the peanut consumption group were instructed to consume at least 6 g of peanut protein weekly from baseline until the age of 60 months. Clinical assessments, including skin-prick tests (SPT) and blood draws were performed at baseline, 12, 30 and 60 months of age. Parents of infants reported food allergy and dietary history at baseline; parents also reported adverse events and food reaction history throughout the duration of the study. The study received ethical approval from the NRES Committee London (REC Reference 04/Q0401/13) and informed consent was obtained from the parents of all participants.

### Whole Milk Protein Exposure

Whole milk protein exposure was introduced in the diet either by means of infant formula or solid foods. Infant formula introduction consisting of casein-dominant, whey-dominant, no or low lactose, goat's milk or partial hydrolysate at baseline were considered whole milk introduction. Infant formula introduction consisting of extensively hydrolysate, amino acid, elemental or soya formula were not considered whole milk introduction. Parents reported cumulative time on formula at baseline, which was then used to compute age started infant formula. In addition, parents reported the age at which their infants started cow's milk food (e.g., cheese or yogurt) at baseline, aged 4-11 months. The age of introduction to whole milk protein was determined as the age at which the participant first had intact milk protein formula or cow's milk food.

### Milk Allergy Outcomes

Infants underwent cow's milk skin prick tests (SPT) and cow's milk specific IgE at baseline (4-11 months of age), 12, 30 and 60 months of age. SPTs were performed using Soluprick® extracts (ALK-Abello) using both a positive and negative control (histamine and saline 0.9%, respectively). In general, SPTs were performed on the forearm. In the event that the entire forearm was covered in eczematic lesions, SPT was performed on the back. The inventors defined a wheal diameter >3 mm or cow's milk specific IgE ≥0.35 kU/L as milk sensitized.

Parent-reported milk allergy was collected at baseline only. Post-baseline throughout the study period, adverse events and food reaction history data were used to identify clinically significant food-induced immediate-onset allergic reactions, and adverse events where system organ class was classified as immune system disorders and preferred terms indicated milk allergy.

### Maternal Peanut Protein Consumption

Mothers of participants reported peanut protein consumption information at baseline, which included information on maternal peanut consumption during pregnancy and breastfeeding. Consumption information was recorded as grams per week.

### Peanut Allergy Outcomes

Infants underwent peanut SPTs at baseline (4-11 months of age), and at 12, 30 and 60 months of age. SPTs were performed using SOLUPRICK® extracts (ALK-Abello) using both a positive and negative control (histamine and saline 0.9%, respectively). In general, SPTs were performed on the forearm. In the event that the entire forearm was covered in eczematic lesions, SPT was performed on the back. The inventors defined a wheal diameter ≥3 mm as peanut sensitized. Participants underwent a 5 g cumulative OFC at 60 months of age (a binary indicator) to assess peanut allergy.

### Sensitivity Analysis

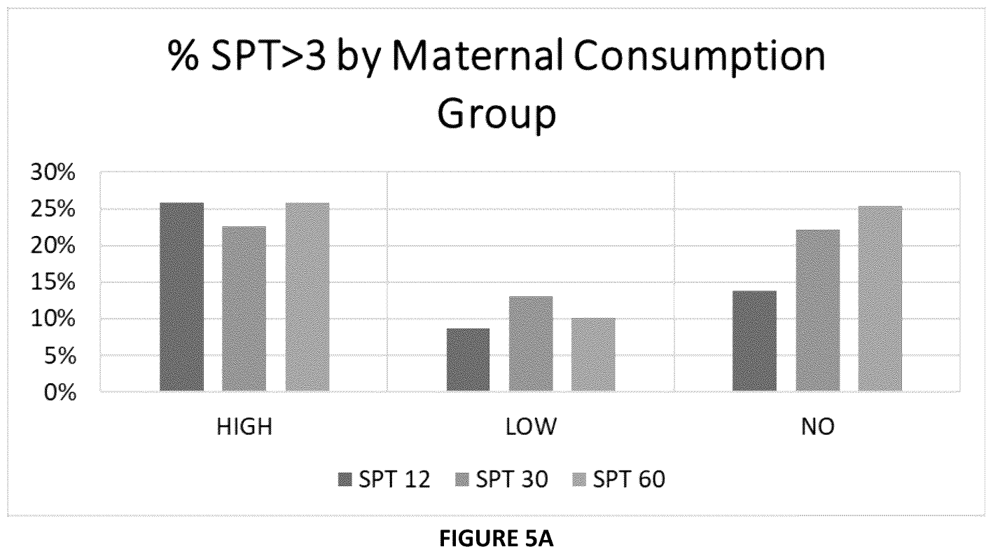

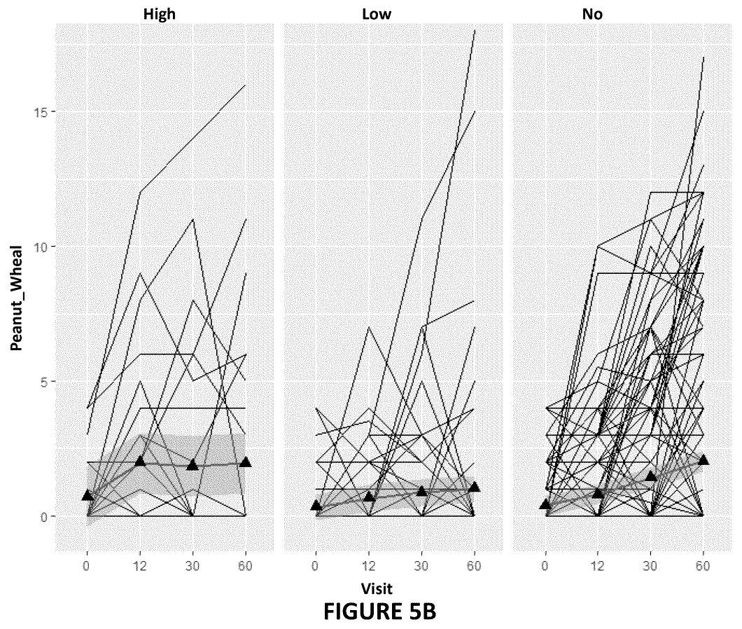

The maternal peanut consumption thresholds presented in this study were assigned according to the distribution of the data as shown in FIG. 5. The greater portion of the study population that consumed peanuts consumed in the range of 0 and 5 gr/week (mean=5). These boundaries were used to describe the “Low” consumption group. Any value greater than 5 gr was considered as “High” consumption.

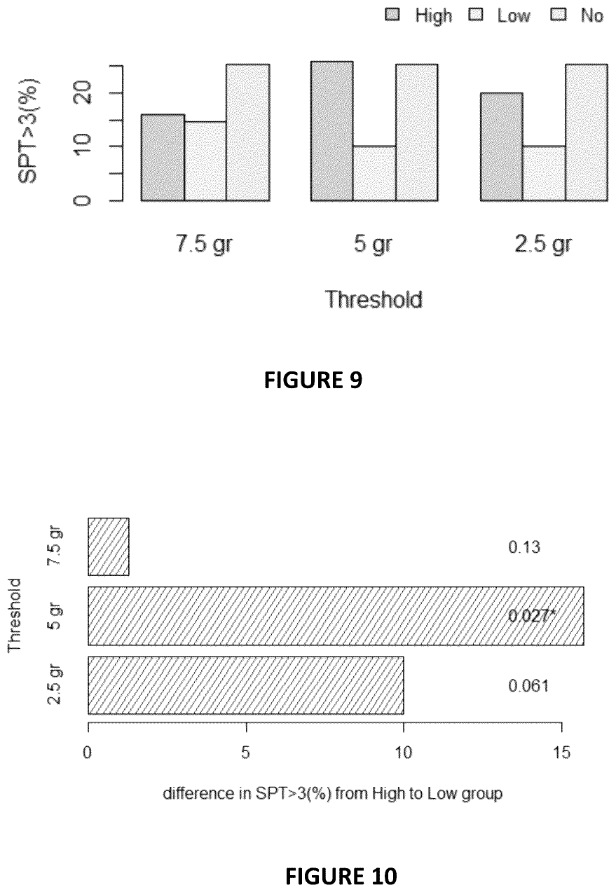

The inventors used segmented regression to explore potential thresholds and found two other potential thresholds between low and high group, according to regression trends: 7.5 gr/week and 2.5 gr/week. Results in Tables 1-2 and FIGS. 9-10, show that the measurement of separation between groups is most significant at threshold 5 gr. Of importance, despite losing statistical significance at 7.5 gr/week and 2.5 gr/week thresholds, the low consumption group consistently presents with the lowest percentage of peanut allergic and peanut sensitized children at 60 months of age.

TABLE 1
SPT ≥ 3 at 60 m at different peanut consumption thresholds
SPT at 60 m

| Threshold | High | | | Low | | | No | | p- |
|---|---|---|---|---|---|---|---|---|---|
| (gr) | N | SPT > 3(%) | | N | SPT > 3(%) | | N | SPT > 3(%) | value |
| 7.5 | 25 | 4 | (16) | 75 | 11 | (14.7) | 181 | 46 (25.4) | 0.13 |
| 5 | 31 | 6 | (25.8) | 69 | 7 | (10.1) | 181 | 46 (25.4) | 0.027* |
| 2.5 | 50 | 10 | (20) | 50 | 5 | (10) | 181 | 46 (25.4) | 0.061 |

TABLE 2
OFC Positive at 60 m at different peanut consumption thresholds
OFC at 60 m

| Threshold | High | | Low | | No | | |
|---|---|---|---|---|---|---|---|
| (gr) | N | SPT > 3(%) | N | SPT > 3(%) | N | SPT > 3(%) | p-value |
| 7.5 | 25 | 3 (12) | 75 | 8 (10.7) | 181 | 34 (18.8) | 0.23 |
| 5 | 31 | 6 (19.4) | 69 | 5 (7.2) | 181 | 34 (18.8) | 0.073 |
| 2.5 | 50 | 8 (16) | 50 | 3 (6) | 181 | 34 (18.8) | 0.093 |

*P-Value < .05

### Statistical Analysis

The inventors used a chi-square test for testing the association between the outcome, i.e., SPT at 60 months, and the exposure, i.e., maternal peanut consumption, categorized into three groups (No, Low, High). ANOVA was used to test the differences in continuous variables (e.g., birthweight) in different exposure groups. The monotonic relationship between peanut exposure routes was assessed using Pearson's Correlation Coefficient. A generalized linear mixed effects model for repeated measures was used to model the repeated measurements of SPT (an outcome in this model) at different time points. The model includes fixed effects for maternal peanut consumption interacted with time (age) of measurements (12, 30 and 60 months) and a random intercept accounting for the within patient correlation, along with the baseline fixed effects for gender, age at enrollment, type of birth and gestational age, and baseline SPT value. A logistic multivariate regression with lasso regularization model was used to predict the probability of peanut sensitization (SPT) at age 60 months, using information collected at baseline. The robustness of the analysis was checked with 2 sensitivity analyses addressing different cutoffs for maternal consumption while breastfeeding. Results with p-values less than 0.05 were considered statistically significant. All analyses were performed using the R statistical programming language (version 4.1.2).

### Ethics Statement

This study was performed according to the approved protocols and procedures received from the Helsinki Committee of Leumit Healthcare Services, Israel (approval number 0026-22-LEU). A written informed consent waiver was obtained by the Helsinki committee based on the nature of the study, specifically the use of deidentified and previously collected data.

### Data Sources

The inventors performed a retrospective, cross-sectional database study from patients with medical records stored in the Leumit Health Services (Leumit) Electronical Medical Record (EMR). Leumit is one of four national healthcare insurers and providers and provides coverage to roughly 700,000 members throughout Israel. Leumit's system has electronically captured routine data from all medical consultations, hospitalizations, laboratory tests, procedures, prescriptions, and other sociodemographic data provided through the Leumit network since 1999, and data is available for research purposes from 2003 and onwards.

All data was anonymized by Leumit's Research and Innovation data team and were made accessible to the researchers only post-anonymization. HebSafeHarbor, an advanced anonymization and deidentification program which automatically identifies personal health information and renders the text unreadable, was applied to all free text data to ensure anonymization of the data⁶.

### Subjects, Cohorts, and Clinical Variables

The FA population (n=4,077) included pediatric patients born after 2010 with an initial FA diagnosis by an allergist before the age of four. ICD-9 codes indicative of a FA diagnosis include: V15.01, V15.02, V15.03, V15.04, V15.05, 995.3, 995.60, 995.61, 995.63, 95.64, 995.67 and 693. All relevant codes and their explanation are presented in Table 12.

The non-FA control population (n=95,686) was selected from the general population and included individuals who were never diagnosed with FA and met the same criteria as the FA population. The control population was matched by year of birth to the FA population. Raw clinical variables included gender, date of birth, number of siblings, diagnostic codes indicative of atopic conditions for siblings, parents, and the infant, and prescriptions and purchases of medications in the prenatal and post-natal period. Clinical variables are further described in Table 11 and a list of antibiotics which were prescribed to either the mother or infant is presented in Table 13. All variables were derived from the prenatal and postnatal period prior to the food allergy diagnosis, but no later than from 4 months of age.

### Statistical Methodology

The inventors used a chi-square test for testing the association between the outcome, FA diagnosis by an allergist, and the various risk factors presented in Table 11. T-test was used to test the differences in independent continuous variables in different groups. The monotonic relationship between FA and explanatory variables was assessed using Pearson's Correlation Coefficient. Logistic multivariate regression was used to analyze the infant's risk factors for AD and generate the Odd's Ratios and Confidence Intervals presented in Table 1. Results with p-values less than 0.05 were considered statistically significant. All analyses were performed using the R statistical programming language (version 4.2.2) and Python programming language.

### Random Forest Regression Models

A random forest regressor algorithm was used to predict the probability of an infant developing FA up to 4 years of age. The random forest regressor technique is an ensemble technique of parallel computation by randomly sampling rows and features (bootstrapping) and aggregating all separate results. 70% of the data was used to train the model, and 30% to test the model performance. A 5-folds cross-validation was performed for model validation. Python's Sklearn package was used for model development and validation.

### Example 1

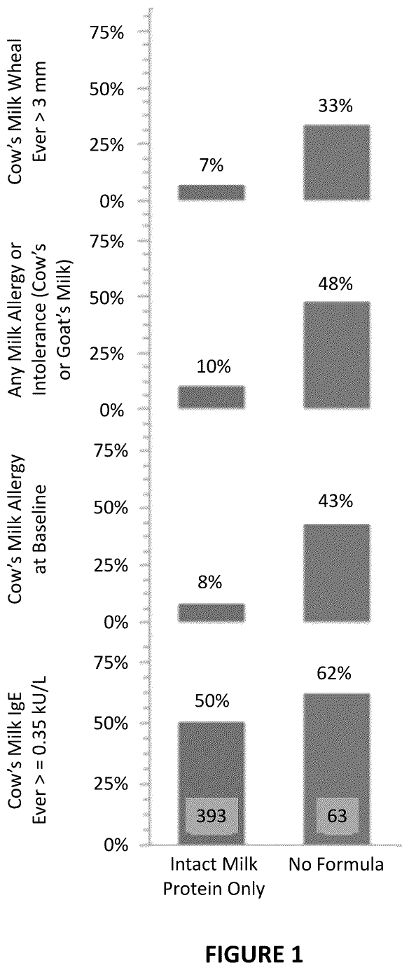

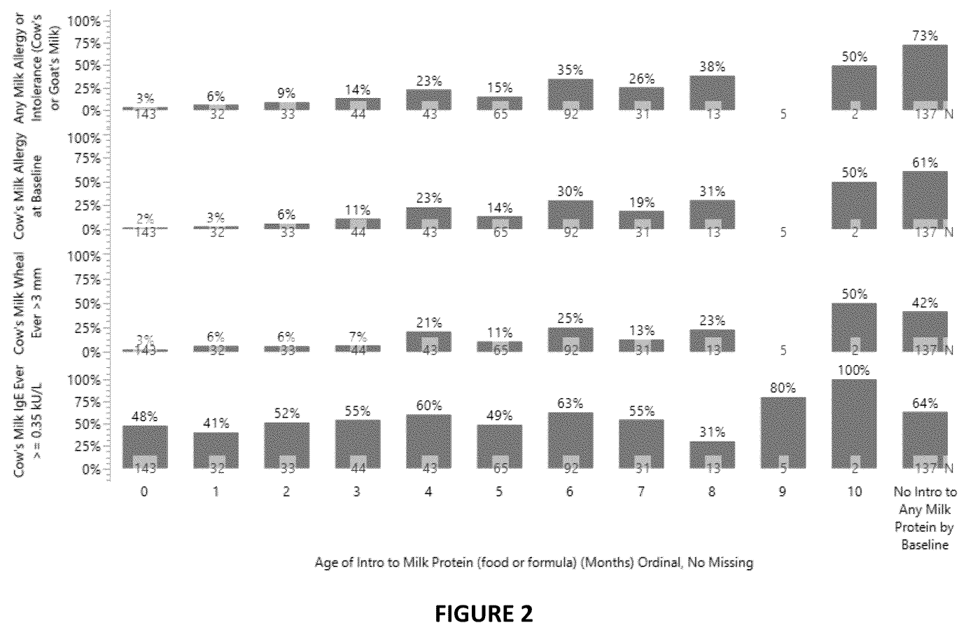

Infants consuming intact milk formula protein demonstrated a 7% prevalence of milk protein sensitivity as measured by skin prick tests, whereas those not given the formula demonstrated a 33% prevalence (FIG. 1). Furthermore, for each month's delay in introduction of intact milk protein, in the form of infant formula, a higher prevalence of milk allergy, sensitivity and or intolerance was observed (FIG. 2). The greatest preventative effect was noticeable with the introduction of intact milk protein formula in the first three month of life (9%) as opposed to afterwards (>14%).

### Example 2

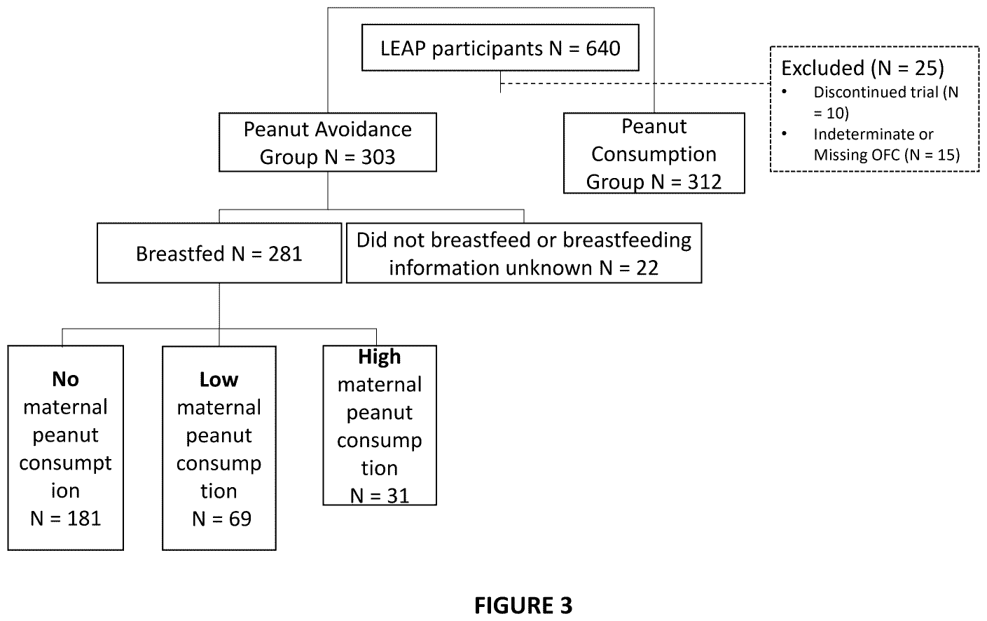

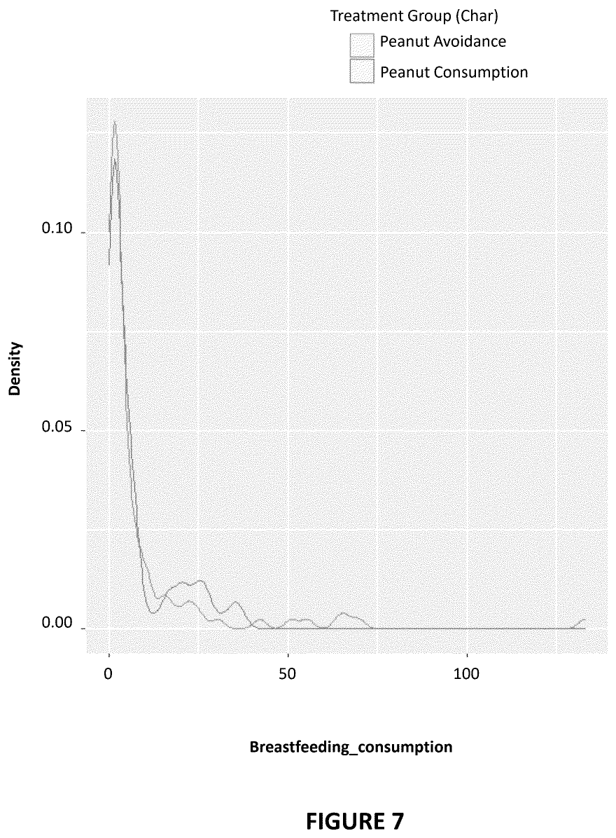

Of the 640 infants randomized into the trial, 615 completed the trial and underwent both SPT & OFC assessments for peanuts at 60 months of age without inconclusive results. Three hundred and twelve (312) of the 615 patients were in the consumption group and 303 were in the avoidance, or control, group (FIG. 3). No significant differences in maternal peanut consumption throughout the breastfeeding period were noted between the consumption and avoidance groups (FIG. 7).

TABLE 3
Maternal consumption of peanuts in the LEAP study cohort

| | | Peanuts | Peanuts | |
|---|---|---|---|---|
| | | avoidance, | consumption, | |
| | | N = 303 | N = 312 | P-value |
| Patient without | N(%) | 203 (67%) | 220 (71%) | |
| maternal | | | | |
| consumption | | | | |
| of peanuts | | | | |
| Patient with | N(%) | 100 (33%) | 92 (29%) | |
| maternal | Median (IQR), | 2.55 | 3.28 | 0.48 |
| consumption | (Total | [1.15,7.58] | [1.30,7.60] | |
| of peanuts | grams/week) | | | |

Comparison of maternal consumption of peanuts throughout breastfeeding in the control and study arms of the LEAP trial. No significant differences were noticed between the two groups with regards to maternal consumption of peanuts.

The inventors performed all analyses in this study on the avoidance group only, as the early and sustained consumption of peanuts in the first 11 months of life significantly reduced the proportion of infants with peanut sensitization or allergy. Therefore, the control group provided an opportunity to assess the influence of maternal consumption without the influence of infant-consumption. The inventors divided the 303 infants included in this analysis into three groups; those whose mothers did not consume peanut (“No”), those whose mothers consumed up to 5 g peanuts per week (“Low”) and those whose mothers consumed more than 5 g of peanuts per week (“High”) (FIG. 3).

### Example 3

The median amount of maternal peanut protein consumed in the avoidance group while lactating was 2.55 g/week including mothers who consumed peanuts. The environmental, birth, ethnicity, eczema, breastfeeding, and baseline SPT values did not differ substantially between the three maternal consumption groups (Table 4).

TABLE 4
Participant characteristics

| | | High | | Low | | No | | | P- |
|---|---|---|---|---|---|---|---|---|---|
| | | N = 31 | | N = 69 | | N = 181 | | Missing | Value |
| Age at baseline (mean(SD)) | months | 7.27 | (1.80) | 7.93 | (1.67) | 7.97 | (1.70) | 0 | 0.11 |
| Season of birth (%) | | | | | | | | 0 | 0.49 |
| | Autumn | 7 | (22.6) | 22 | (31.9) | 59 | (32.6) | | |
| | Summer | 5 | (16.1) | 11 | (15.9) | 34 | (18.8) | | |
| | Spring | 12 | (38.7) | 24 | (34.8) | 44 | (24.3) | | |
| | Winter | 7 | (22.6) | 12 | (17.4) | 44 | (24.3) | | |
| Gender | Female | 11 | (35.5) | 23 | (33.3) | 70 | (38.7) | 0 | 0.72 |
| | (%) | | | | | | | | |
| Birthweight (mean(SD)) | kg | 3.49 | (0.73) | 3.48 | (0.47) | 3.56 | (0.49) | 0 | 0.54 |
| Gestational Age (mean(SD)) | Weeks | 39.55 | (1.77) | 39.81 | (1.60) | 40.03 | (1.45) | 0 | 0.21 |
| Type of Birth | Vaginal | 18 | (58.1) | 50 | (72.5) | 133 | (73.5) | 0 | 0.21 |
| | (%) | | | | | | | | |
| Ethnicity | White | 21 | (67.7) | 52 | (75.4) | 139 | (76.8) | 0 | 0.56 |
| | (%) | | | | | | | | |
| Mother smoked during | Yes(%) | 2 | (6.5) | 3 | (4.3) | 6 | (3.3) | 0 | 0.69 |
| pregnancy | | | | | | | | | |
| Daycare | Yes(%) | 3 | (9.7) | 9 | (13.0) | 30 | (16.6) | 0 | 0.54 |
| Cats in the house | Yes(%) | 3 | (9.7) | 13 | (18.8) | 19 | (10.5) | 0 | 0.18 |
| Dogs in the house | Yes(%) | 2 | (6.5) | 2 | (2.9) | 10 | (5.5) | 0 | 0.64 |
| Vitamin D (mean(SD)) | | 0.21 | (0.86) | 0.09 | (0.32) | 0.21 | (1.07) | 19 | 0.66 |
| Peanut SPT > 0 at Baseline | Yes(%) | 9 | (29.0) | 9 | (13.0) | 28 | (15.5) | 0 | 0.12 |
| SCORAD Group (%) | | | | | | | | 0 | 0.26 |
| | <15 | 5 | (16.1) | 9 | (13.0) | 31 | (17.1) | | |
| | >40 | 8 | (25.8) | 21 | (30.4) | 72 | (39.8) | | |
| | 15-40 | 18 | (58.1) | 39 | (56.5) | 78 | (43.1) | | |
| Breastfed | Yes(%) | 30 | (96.8) | 69 | (100.0) | 178 | (98.3) | 0 | 0.41 |
| Duration of Breastfeeding | | | | | | | | 5 | 0.16 |
| (%) | | | | | | | | | |
| | 1 month | 2 | (6.7) | 2 | (2.9) | 20 | (11.2) | | |
| | 3 | 5 | (16.7) | 4 | (5.8) | 25 | (14.0) | | |
| | months | | | | | | | | |
| | 6 | 10 | (33.3) | 25 | (36.2) | 55 | (30.9) | | |
| | months | | | | | | | | |
| | over 6 | 13 | (43.3) | 38 | (55.1) | 78 | (43.8) | | |
| | months | | | | | | | | |
| Exclusively Breastfed at | Yes(%) | 24 | (77.4) | 60 | (87.0) | 154 | (85.1) | 0 | 0.46 |
| Baseline | | | | | | | | | |
| Milk SPT at Baseline > 3 | Yes(%) | 6 | (19.4) | 8 | (11.6) | 39 | (21.5) | 0 | 0.20 |
| Raw Egg SPT at | Yes(%) | 19 | (61.3) | 46 | (66.7) | 126 | (69.6) | 0 | 0.63 |
| Baseline > 3 | | | | | | | | | |
| Egg SPT at Baseline > 3 | Yes(%) | 18 | (58.1) | 37 | (53.6) | 107 | (59.1) | 0 | 0.73 |
| Sesame SPT at Baseline > 3 | Yes(%) | 3 | (9.7) | 4 | (5.8) | 16 | (8.8) | 0 | 0.70 |
| Soya SPT at Baseline > 3 | Yes(%) | 0 | (0.0) | 3 | (4.3) | 5 | (2.8) | 0 | 0.48 |
| Maternal Eczema | Yes(%) | 12 | (70.6) | 11 | (30.6) | 56 | (52.8) | 122 | 0.01 |
| Paternal Eczema | Yes(%) | 5 | (45.5) | 18 | (43.9) | 48 | (46.6) | 126 | 0.96 |
| Maternal Asthma | Yes(%) | 9 | (52.9) | 9 | (25.0) | 28 | (26.4) | 122 | 0.07 |
| Paternal Asthma | Yes(%) | 5 | (45.5) | 11 | (26.8) | 40 | (38.8) | 126 | 0.32 |
| Maternal | Yes(%) | 10 | (58.8) | 19 | (52.8) | 59 | (55.7) | 122 | 0.91 |
| Rhinoconjunctivitis | | | | | | | | | |
| Paternal Rhinoconjunctivitis | Yes(%) | 6 | (54.5) | 24 | (58.5) | 63 | (61.2) | 126 | 0.89 |
| Maternal Food Allergy | Yes(%) | 3 | (17.6) | 5 | (13.9) | 15 | (14.2) | 122 | 0.92 |
| Paternal Food Allergy | Yes(%) | 0 | (0.0) | 7 | (17.1) | 17 | (16.5) | 126 | 0.34 |

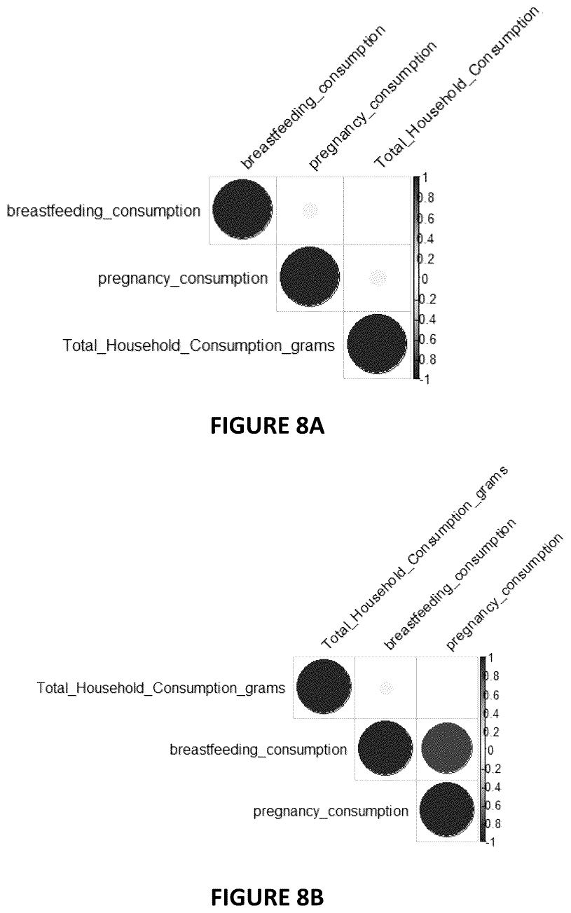

While self-reported maternal history of eczema differed significantly between the two groups, 43% of 303 participants did not provide maternal history of eczema in the publicly available dataset. The median amount of maternal peanut consumption in the low consumption group was 1.45 g/week (IQR 0.79, 2.60) and in the high consumption group it was 16.10 g/week (IQR 7.50, 26.11). Peanut consumption while pregnant and while breastfeeding was significantly correlated (r=0.87, FIG. 8, whereas total household consumption of peanuts was not correlated with either maternal consumption periods (r=0.08 for breastfeeding consumption & r=0.06 for consumption during pregnancy).

### Example 4

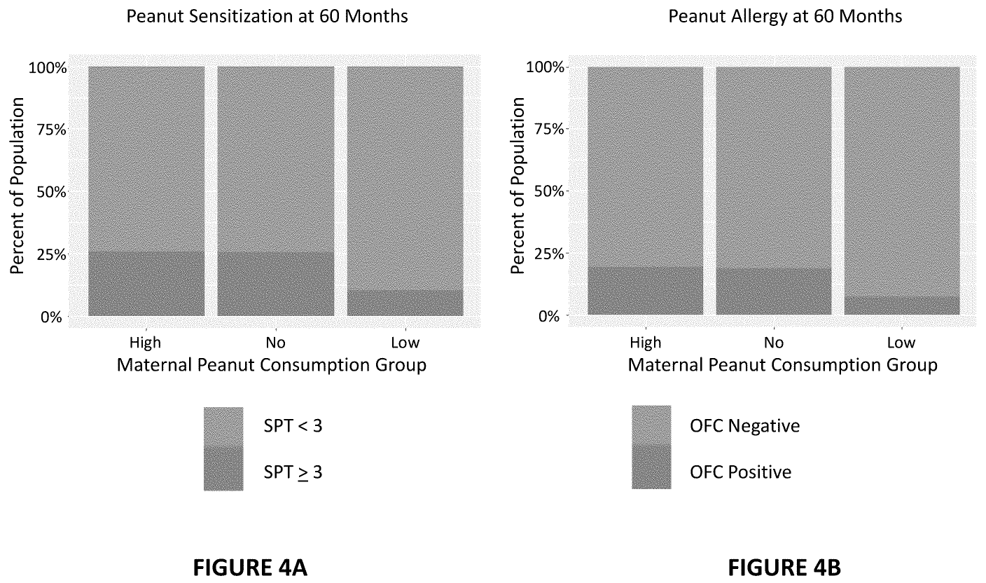

Both peanut allergy outcomes, sensitization (SPT) and allergy (OFC), occurred less frequently at 60 months of age in infants whose mothers consumed a moderate amount of peanuts while breastfeeding when compared to those who either did not consume peanuts while breastfeeding or those who consumed a large amount of peanuts when breastfeeding (FIG. 4). Twenty-five-point eight percent (25.8%) of children of high consumers, 10.1% of children of moderate consumers and 25.4% of children of non-consumers had a peanut SPT≥3 (p=0.027) and 19.4% of children of high consumers, 7.2% of children of moderate consumers and 18.8% of children of non-consumers had a positive peanut OFC (p=0.073) at 60 months of age (FIG. 4). There are three known routes of exposure to peanut protein prior to initiation of solid feeding; maternal consumption while pregnant, maternal consumption while breastfeeding and environmental exposure. A univariate analysis for the three routes of exposure to peanuts in our study population showed that low maternal consumption of peanuts while breastfeeding significantly reduces peanut sensitization at 60 months of age compared to the “High” and “No” groups (Table 5).

TABLE 5
Univariate analysis for external exposure to peanuts in the control group

| | | OR | CI | P-Value |
|---|---|---|---|---|
| Consumption while Breastfeeding | Low | 0.32 | 0.1-1 | 0.049* |
| | No | 0.98 | 0.42-2.47 | 0.963 |

| | | | |
|---|---|---|---|
| Consumption while Pregnant | 1 | 0.99-1 | 0.58 |
| Household Consumption | 1 | 0.98-1.01 | 0.957 |

*High is the reference category

The inventors used mixed effects models to estimate the effects of time and maternal peanut consumption while breastfeeding on peanut sensitization. A logistic mixed effects model for a binary outcome indicator of SPT≥3 was fitted. The results shown in Table 3 suggest that the marginal effect of the “Low” consumption group leads to decreased sensitization risk in comparison to the “High” consumption group (OR 0.12, P=0.058).

TABLE 6
Generalized mixed effects model for repeated measures of SPT

| | | | | | | Lower | Upper |
|---|---|---|---|---|---|---|---|
| | Estimate | Std. Error | Pr(>\|z\|) | | OR | bound | bound |
| (Intercept) | −0.49472 | 6.28803 | 0.9373 | | | | |
| Stratum | 3.33624 | 0.68975 | <0.0001 | *** | 28.11 | 7.27 | 108.65 |
| (SPT >0 at | | | | | | | |
| baseline) | | | | | | | |
| Sex (Female) | −0.81759 | 0.50823 | 0.1077 | | 0.44 | 0.16 | 1.20 |
| Age | −0.08128 | 0.13974 | 0.5608 | | 0.92 | 0.70 | 1.21 |
| Type of birth | −0.20448 | 0.51894 | 0.6936 | | 0.82 | 0.29 | 2.25 |
| (Vaginal) | | | | | | | |
| Gestational | −0.01202 | 0.172 | 0.9443 | | 0.99 | 0.71 | 1.38 |
| age | | | | | | | |
| Birth Weight | −0.35736 | 0.53176 | 0.5016 | | 0.70 | 0.25 | 1.98 |
| Low | −2.1185 | 1.11932 | 0.0584 | . | 0.12 | 0.01 | 1.08 |
| Maternal | | | | | | | |
| Consumption | | | | | | | |
| at | | | | | | | |
| Breastfeeding | | | | | | | |
| No Maternal | −1.43352 | 0.95431 | 0.1331 | | 0.24 | 0.04 | 1.55 |
| Consumption | | | | | | | |
| at | | | | | | | |
| Breastfeeding | | | | | | | |
| 30 months | −0.56616 | 0.8825 | 0.5212 | | 0.57 | 0.10 | 3.20 |
| 60 months | −0.22649 | 0.85745 | 0.7917 | | 0.80 | 0.15 | 4.28 |
| Low Maternal | 1.51842 | 1.16868 | 0.1939 | | 4.57 | 0.46 | 45.11 |
| Consumption at | | | | | | | |
| Breastfeeding: 30 | | | | | | | |
| No Maternal | 1.893 | 0.98924 | 0.0557 | . | 6.64 | 0.96 | 46.15 |
| Consumption at | | | | | | | |
| Breastfeeding: 30 | | | | | | | |
| Low Maternal | 0.66137 | 1.16428 | 0.57 | | 1.94 | 0.20 | 18.98 |
| Consumption at | | | | | | | |
| Breastfeeding: 60 | | | | | | | |
| No Maternal | 1.84418 | 0.96411 | 0.0558 | . | 6.32 | 0.96 | 41.84 |
| Consumption at | | | | | | | |
| Breastfeeding: 60 | | | | | | | |

The interaction between maternal consumption groups while breastfeeding and the time indicates that not consuming peanuts while breastfeeding has a negative effect on the infant's sensitization to peanuts measured by a larger SPT over time. This finding is illustrated in FIG. 5.

### Example 5

### Factors Contributing to Peanut Sensitization at 60 Months

In an effort to discern the leading contributing factors to peanut sensitization at 60 months of age, the inventors fitted a logistic regression model with lasso regularization. The inventors incorporated all the baseline factors, controlling demographic, clinical and family history variables. All 303 participants from the control arm of the LEAP trial were included. This cohort was randomly split into a training and a validation set in a 70/30 ratio. Table 7 compares the demographic and clinical characteristics of the training and validation sets. Importantly, no significant differences were noted between the training and validation set in any of the characteristics.

TABLE 7
Characteristics of training & test sets

| | | Test, | Train, | | P- |
|---|---|---|---|---|---|
| | | N = 91 | N = 212 | Missing | Value |
| Age at baseline | months | 7.93 (1.7) | 7.82 (1.71) | 0 | 0.62 |
| (mean(SD)) | | | | | |
| Season of | | | | 0 | 0.17 |
| birth (%) | | | | | |
| | Autumn | 25 (27.5) | 67 (31.6) | | |
| | Summer | 11 (12.1) | 43 (20.3) | | |
| | Spring | 33 (36.3) | 56 (26.4) | | |
| | Winter | 22 (24.2) | 46 (21.7) | | |
| Gender | Female (%) | 30 (33.0) | 78 (36.8) | 0 | 0.61 |
| Birthweight | | 3.58 (0.51) | 3.53 (0.53) | 0 | 0.49 |
| (mean(SD)) | | | | | |
| Gestational Age | | 39.91 | 39.92 | 0 | 0.95 |
| (mean(SD)) | | (1.41) | (1.60) | | |
| Type of Birth | Vaginal (%) | 61 (67.0) | 154 (72.6) | 0 | 0.40 |
| Ethnicity | White (%) | 69 (75.8) | 163 (76.9) | 0 | 0.96 |
| Mother smoked | Yes(%) | 3 (3.3) | 9 (4.2) | 0 | 0.95 |
| during pregnancy | | | | | |
| Daycare | Yes(%) | 12 (13.2) | 36 (17.0) | 0 | 0.51 |
| Cats in the house | Yes(%) | 16 (17.6) | 24 (11.3) | 0 | 0.20 |
| Dogs in the house | Yes(%) | 6 (6.6) | 12 (5.7) | 0 | 0.96 |
| Vitamin D | | 0.07 (0.24) | 0.21 (1.05) | 19 | 0.20 |
| (mean(SD)) | | | | | |
| SPT > 0 at | Yes(%) | 16 (17.6) | 32 (15.1) | 0 | 0.71 |
| Baseline | | | | | |
| Scorad Group (%) | | | | 0 | 0.41 |
| | <15 | 13 (14.3) | 36 (17.0) | | |
| | >40 | 30 (33.0) | 82 (38.7) | | |
| | 15-40 | 48 (52.7) | 94 (44.3) | | |
| Breastfed | Yes(%) | 82 (90.1) | 196 (92.5) | 0 | 0.65 |
| Duration of | | | | 25 | 0.05 |
| Breastfeeding (%) | | | | | |
| | 1 month | 10 (12.2) | 15 (7.7) | | |
| | 3 months | 5 (6.1) | 29 (14.8) | | |
| | 6 months | 22 (26.8) | 68 (34.7) | | |
| | over 6 | 45 (54.9) | 84 (42.9) | | |
| | months | | | | |
| Exclusively | Yes(%) | 70 (76.9) | 168 (79.2) | 0 | 0.77 |
| Breastfed at | | | | | |
| Baseline | | | | | |
| Milk SPT at | Yes(%) | 18 (19.8) | 35 (16.5) | 0 | 0.60 |
| Baseline >3 | | | | | |
| Row Egg SPT | Yes(%) | 58 (63.7) | 149 (70.3) | 0 | 0.32 |
| at Baseline >3 | | | | | |
| Egg SPT at | Yes(%) | 46 (50.5) | 128 (60.4) | 0 | 0.14 |
| Baseline >3 | | | | | |
| Sesame SPT | Yes(%) | 6 (6.6) | 17 (8.0) | 0 | 0.85 |
| at Baseline >3 | | | | | |
| Soy SPT at | Yes(%) | 1 (1.1) | 7 (3.3) | 0 | 0.48 |
| Baseline >3 | | | | | |
| Maternal Eczema | Yes(%) | 26 (45.6) | 60 (52.2) | 131 | 0.52 |
| Paternal Eczema | Yes(%) | 23 (39.0) | 53 (50.5) | 139 | 0.21 |
| Maternal Asthma | Yes(%) | 15 (26.3) | 36 (31.3) | 131 | 0.62 |
| Paternal Asthma | Yes(%) | 19 (32.2) | 39 (37.1) | 139 | 0.64 |
| Maternal | Yes(%) | 32 (56.1) | 63 (54.8) | 131 | 1.00 |
| Rhino- | | | | | |
| conjunctivitis | | | | | |
| Paternal | Yes(%) | 33 (55.9) | 64 (61.0) | 139 | 0.64 |
| Rhino- | | | | | |
| conjunctivitis | | | | | |
| Maternal | Yes(%) | 8 (14.0) | 18 (15.7) | 131 | 0.96 |
| Food Allergy | | | | | |
| Paternal | Yes(%) | 12 (20.3) | 13 (12.4) | 139 | 0.26 |
| Food Allergy | | | | | |

TABLE 8
SPT outcomes and maternal peanut consumption while
breastfeeding by group at 12, 30 and 60 months

| | High | Low | No | High | Low | No | High | Low | No | |
|---|---|---|---|---|---|---|---|---|---|---|
| n | 31 | 67 | 181 | 31 | 68 | 176 | 31 | 69 | 181 | |
| SPT > | 8 | 6 | 25 | 7 | 9 | 40 | 8 | 7 | 46 | 0.006 |
| 3 = | (25.8) | (9.0) | (13.8) | (22.6) | (13.2) | (22.7) | (25.8) | (10.1) | (25.4) | |
| 1(%) | | | | | | | | | | |
| SPT, | 1.97 | 0.65 | 0.78 | 1.84 | 0.85 | 1.40 | 1.94 | 1.00 | 2.02 | <0.001 |
| Peanut | (3.08) | (1.31) | (1.74) | (3.62) | (2.05) | (2.74) | (3.93) | (3.12) | (3.74) | |
| Wheal | | | | | | | | | | |
| (mean | | | | | | | | | | |
| (SD)) | | | | | | | | | | |

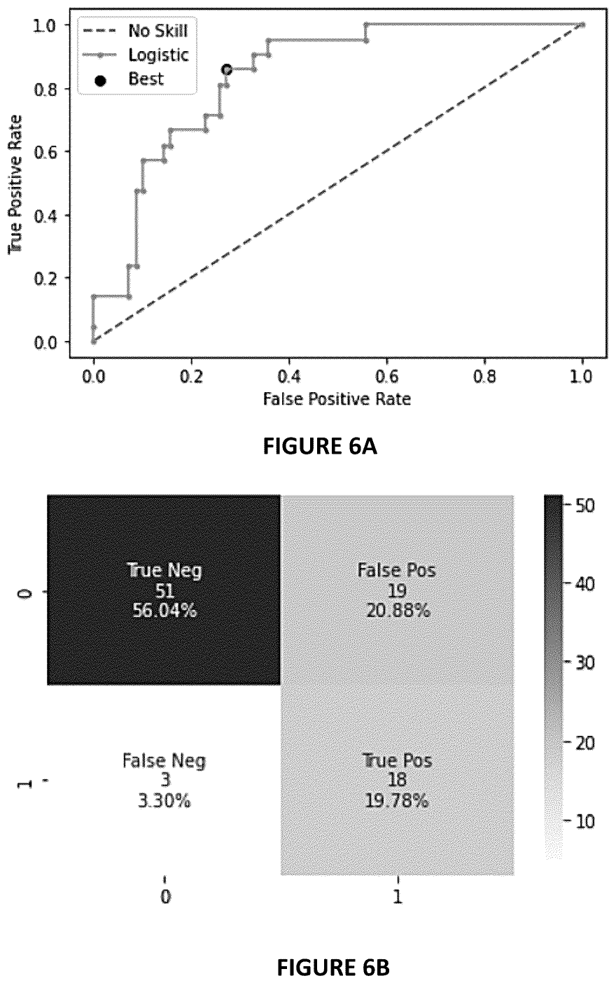

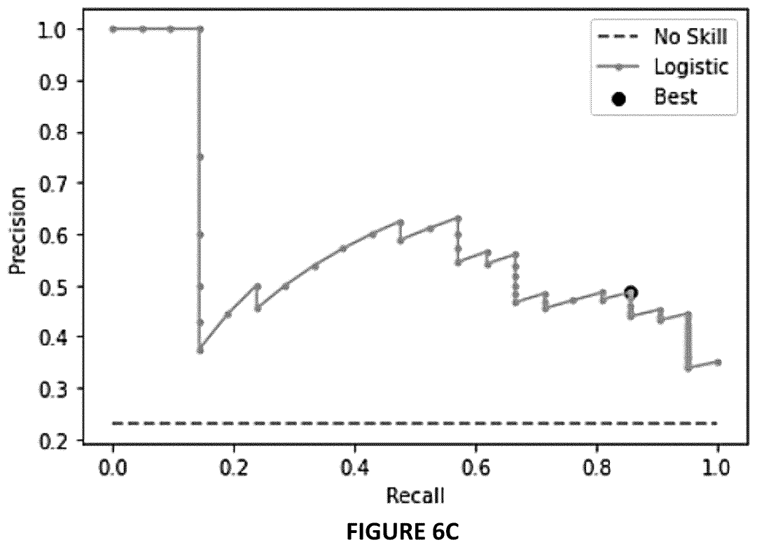

FIG. 6 presents the predictive performance in the validation cohort of the trained model. The model boasts a receiver operating characteristic (ROC) area under the curve (AUC) of 0.79, accuracy of 76%, precision of 49% and recall of 86%. Birth weight, gestational age, type of birth, duration of breastfeeding, daycare attendance, paternal history of asthma, and parental history of eczema, rhinoconjunctivitis or food allergy were not significant contributors to peanut sensitization risk. However, ethnicity (OR 0.47, p=0.046, 95% CI 0.22-0.99), baseline peanut SPT stratum (OR 4.87, p=0.00, 95% CI 2.13-11.12), no maternal peanut consumption while breastfeeding (OR 3.25, p =0.008, 95% CI 1.36-7.77), baseline SCORAD >40 (OR 2.78, p=0.007, 95% CI 1.32-5.85) and maternal asthma (OR 2.87, p=0.013, 95% CI 1.25-6.61) were all significant contributors to peanut sensitization at 60 months of age (Table 9).

TABLE 9
Baseline factors contributing to peanut sensitization at 60 months of age

| | OR | p value | 95% CI | |
|---|---|---|---|---|
| Gender | 0.62 | 0.197 | 0.30 | 1.28 |
| Birthweight | 0.9 | 0.794 | 0.44 | 1.88 |
| Gestational age | 0.94 | 0.177 | 0.86 | 1.03 |
| Type of birth | 0.64 | 0.224 | 0.31 | 1.31 |
| Ethnicity | 0.47 | 0.046 | 0.22 | 0.99 |
| Duration Breastfeeding (months) | 0.92 | 0.153 | 0.81 | 1.03 |
| No Maternal Consumption at | 3.25 | 0.008** | 1.36 | 7.77 |
| Breastfeeding | | | | |
| High Maternal Consumption at | 3.02 | 0.087 | 0.85 | 10.68 |
| Breastfeeding | | | | |
| Baseline Peanut SPT Stratum | 4.87 | 0*** | 2.13 | 11.12 |
| Daycare | 2.19 | 0.075 | 0.92 | 5.22 |
| SCORAD >40 | 2.78 | 0.007** | 1.32 | 5.85 |
| SCORAD <15 | 1.15 | 0.785 | 0.42 | 3.15 |
| Maternal Eczema | 0.63 | 0.238 | 0.29 | 1.36 |
| Paternal Eczema | 0.66 | 0.266 | 0.32 | 1.37 |
| Maternal Asthma | 2.87 | 0.013* | 1.25 | 6.61 |
| Paternal Asthma | 0.59 | 0.216 | 0.26 | 1.36 |
| Maternal Rhinoconjunctivitis | 0.59 | 0.169 | 0.28 | 1.25 |
| Paternal Rhinoconjunctivitis | 1.27 | 0.519 | 0.61 | 2.63 |
| Maternal Food Allergy | 0.89 | 0.801 | 0.38 | 2.11 |
| Paternal Food Allergy | 1.83 | 0.205 | 0.72 | 4.68 |
| Any Wheal greater than = 3 | 2.34 | 0.052 | 0.99 | 5.49 |
| (excluding Peanut) | | | | |

*<.05; **<.01; and ***<.001

### Example 6

The prevalence and burden of food allergy (FA) has grown globally, creating a need to identify infants at risk of food allergies to manage or even prevent the condition ¹. There is no accepted methodology for stratifying one's chances of developing FA, despite numerous studies pointing to genetic ², environmental³ and medical history^{1.3} risk factors for the conditions. A family history of atopic conditions has been used to identify high-risk infants in the immediate post-natal period in the research setting⁴ yet performs poorly and does not allow to distinguish among genetic and environmental risk factors. Electronic medical records (EMR) can be harnessed for risk stratification, as has been done for instance in pediatric obesity⁴. We hypothesized that the combined analysis of risk elements from a large and nationally representative healthcare provider EMR would allow us to develop prediction models and stratify an infant's risk of developing FA.

The inventors performed a retrospective, cross-sectional database study from medical records stored in the Leumit Health Services (Leumit) EMR. Leumit provides coverage and medical services to roughly 700,000 members throughout Israel. Leumit's system has electronically captured routine data from all medical consultations, procedures, prescriptions, and sociodemographic data since 1999.

The FA population (n=4,077) included patients born between 2010 and 2020 with an initial FA diagnosis by an allergist before the age of four. The non-FA control population (n=95,686) was selected from the general population and included individuals who were never diagnosed with FA and were born in the same years. ICD-9 codes used for a FA diagnosis and raw clinical variables are described in Tables 11 and 12. All variables were derived from the prenatal and postnatal period prior to the food allergy diagnosis, but no later than from 4 months of age. Logistic regression and random forest regression models were trained and tested on the combined dataset.

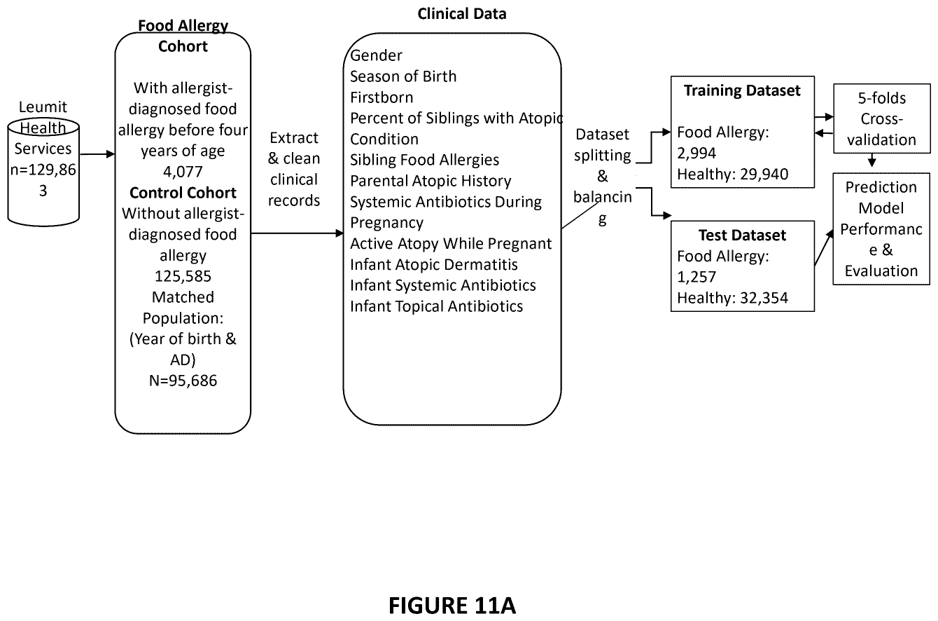

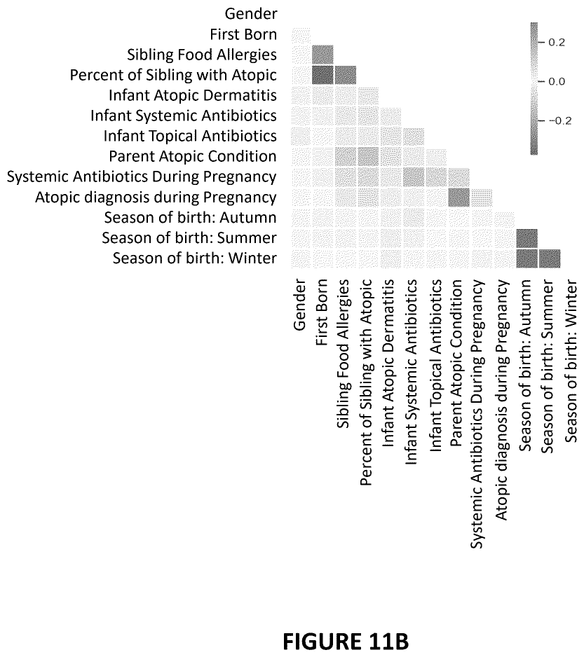

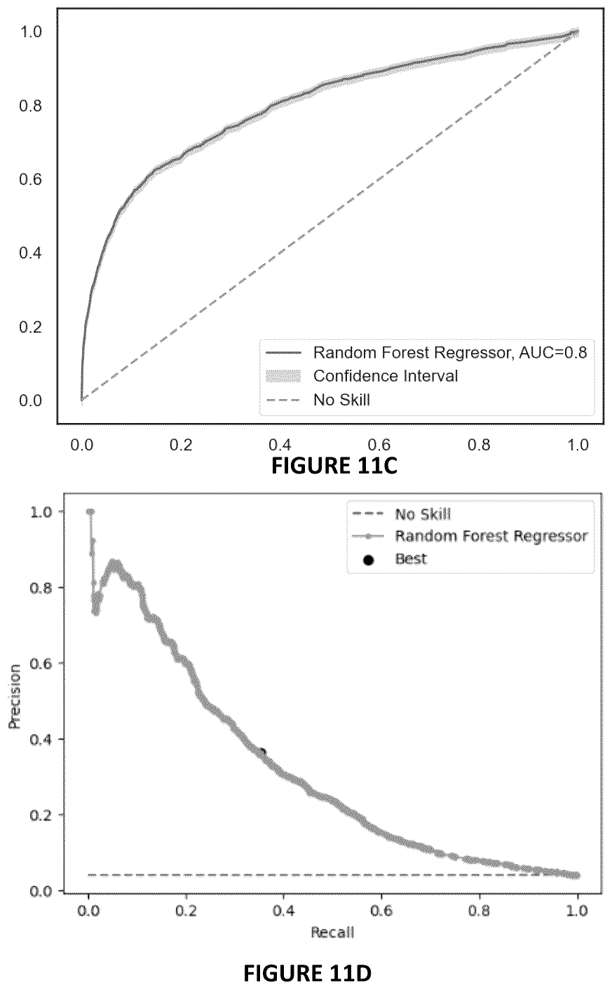

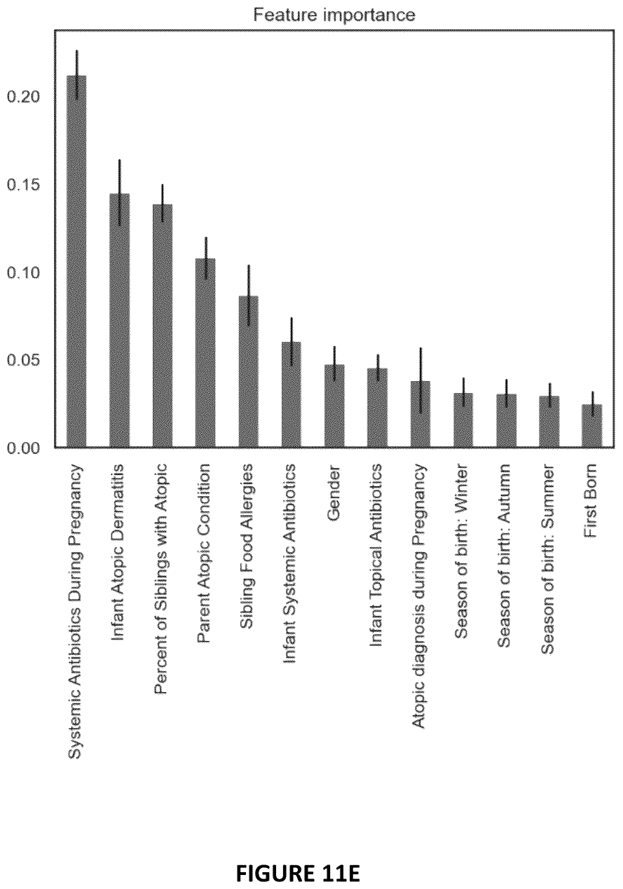

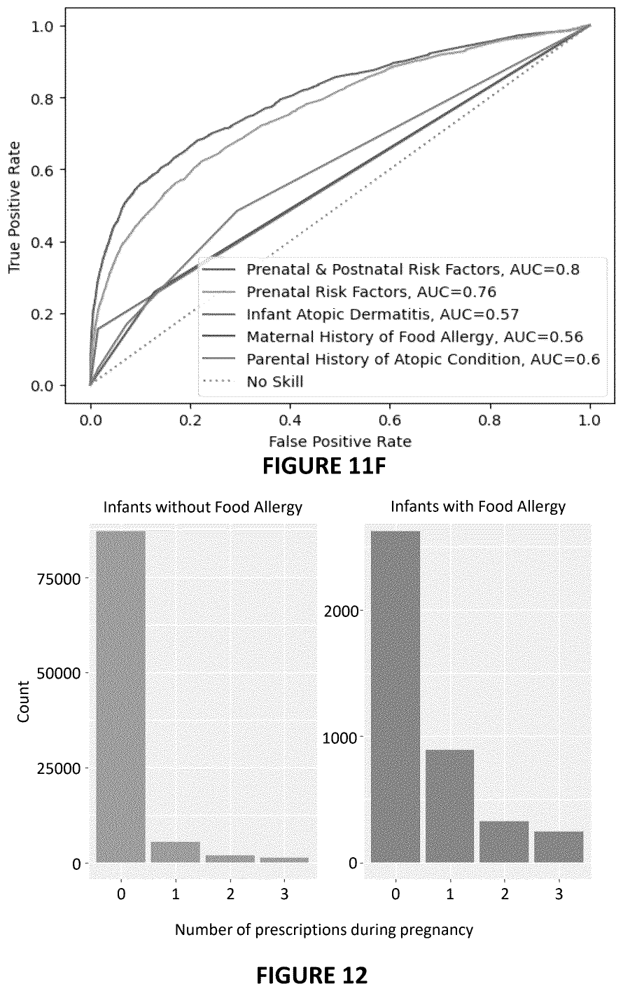

Logistic regression models pointed to several significant risk factors, including the use of systemic antibiotics during pregnancy (OR 1.93, CI 1.82-2, p<0.001) or during infancy (OR 2.86, CI 2.49-3.27, p<0.001), a prior diagnosis of atopic dermatitis (OR 8.61, CI 7.71-9.6, p<0.001) and others (Table 10, and FIG. 11B). Random forest regression models incorporating all risk factors from the period prior to the FA diagnosis resulted in a receiver operating characteristic curve with an area under the curve (AUC) of 0.80 and an accuracy of 83%, with corresponding sensitivity of 62%, and specificity of 84% (FIGS. 11B-11D). When trained using risk factors available only in the prenatal period, the AUC was 0.76 with an accuracy of 79% and corresponding sensitivity of 59%, and a specificity of 80% (FIG. 11F).

Whereas no widely accepted benchmark for the stratification of food allergies exists, we compared our predictive algorithm to parameters that are sporadically employed in the clinical or research setting. These parameters include maternal history of FA, parental history of atopic conditions, or a previous diagnosis of atopic dermatitis before 4 months of age (FIG. 11F). The drastic and significant improvement shown in our regression model demonstrates the model's superiority to a risk assessment using family history or infant history of atopy alone.

Predictive modeling using routinely collected electronic medical record data can serve as a powerful tool to stratify an infant's risk of developing FA. Knowledge of an infant's risk can inform both caregivers and medical professionals as to timely interventions to mitigate the development of FA, including the early introduction of allergens according to local guidelines⁵, and can allow for a drastic reduction in clinical trials designed to assess the efficacy of FA prevention strategies. Future studies incorporating additional data, including maternal and infant dietary practices, are warranted to further hone the predictive capability of these risk stratification techniques.

TABLE 10
Demographic and Clinical Characteristics of Healthy Infants
and Infants with FA with Univariate and Multivariate Analysis

| | Univariate | | | Multivariate | | |
|---|---|---|---|---|---|---|
| | Without FA | With | | | | P- |
| | (N = 95,686) | (N = 4,077) | P-Value | OR | CI | Value |
| Gender = | 49,020 | 2,343 | <0.001 | 1.25 | 1.17-1.33 | <0.001 |
| Male (%) | (51.2) | (57.5) | | | | |
| Season of Birth | | | <0.001 | | | |
| (%) | | | | | | |
| Autumn | 24,381 | 1,147 | | | | |
| | (25.5) | (28.1) | | | | |
| Spring | 22,965 | 927 | | 0.91 | 0.82-0.99 | 0.04 |
| | (24) | (22.7) | | | | |
| Summer | 24,192 | 957 | | 0.91 | 0.83-0.99 | 0.04 |
| | (25.3) | (23.5) | | | | |
| Winter | 24,148 | 1,046 | | 0.94 | 0.86-1.03 | 0.2 |
| | (25.2) | (25.7) | | | | |
| Firstborn = | 30,162 | 1,492 | <0.001 | 2.08 | 1.19-2.26 | <0.001 |
| Yes(%) | (31.5) | (36.6) | | | | |
| Percent of | 0.18 | 0.32 | <0.001 | 2.06 | 1.84-2.3 | <0.001 |
| Siblings with an | (0.33) | (0.41) | | | | |
| Atopic | | | | | | |
| Condition | | | | | | |
| (Mean(SD)) | | | | | | |
| Siblings with | 0.16 | 0.4 | <0.001 | 1.71 | 1.61-1.81 | <0.001 |
| Food Allergy | (0.44) | (0.72) | | | | |
| (Mean(SD)) | | | | | | |
| Parental Atopic | 0.39 | 0.67 | <0.001 | 1.31 | 1.25-1.36 | <0.001 |
| History(Mean(SD)) | (0.68) | (0.85) | | | | |
| Systemic | 0.14 | 0.56 | <0.001 | 1.93 | 1.82-2 | <0.001 |
| Antibiotics | (0.49) | (0.88) | | | | |
| While Pregnant | | | | | | |
| (Mean(SD)) | | | | | | |
| Active Atopy | 0.03 | 0.07 | <0.001 | 1.07 | 0.96-1.18 | 0.23 |
| While Pregnant | (0.23) | (0.35) | | | | |
| (Mean(SD)) | | | | | | |
| Infant Atopic | 1,437 | 599 | <0.001 | 8.61 | 7.71-9.6 | <0.001 |
| Dermatitis = | (1.5) | (14.7) | | | | |
| Yes(%) | | | | | | |
| Infant Systemic | 1,582 | 337 | <0.001 | 2.86 | 2.49-3.27 | <0.001 |
| Antibiotics = | (1.7) | (8.3) | | | | |
| Yes(%) | | | | | | |
| Infant Topical | 754 | 234 | <0.001 | 3.87 | 3.26-4.58 | <0.001 |
| Antibiotics = | (0.8) | (5.7) | | | | |
| Yes(%) | | | | | | |

Abbreviations: OR, odds ratio;
CI, confidence interval

TABLE 11
Summary of independent and dependent variables
of interest in the study

| Variable | Type | Level | Description | Timeline |
|---|---|---|---|---|
| Gender | Dichotomous | Male/Female | Gender | Prenatal |
| Season of | Categorical | Autumn/Spring/ | Season of Birth | Prenatal |
| Birth | | Summer/Winter | | |
| Firstborn | Dichotomous | Yes/No | Firstborn | Prenatal |
| Percent of | Continuous | Fractional in | Number of | Prenatal |
| Siblings | | range 0-100 | siblings with an | |
| with an | | | atopic condition | |
| Atopic | | | divided by the | |
| Condition | | | total number of | |
| | | | siblings | |
| Siblings | Continuous | Integer in | Number of | Prenatal |
| with Food | | range 0-5 | siblings with a | |
| Allergy | | | food allergy | |
| Parental | Continuous | Integer in | Total number of | Prenatal |
| Atopic | | range 0-8 | atopic conditions | |
| History | | | diagnosed in | |
| | | | either parent's | |
| | | | medical file | |
| Systemic | Continuous | Integer in | Number of | Prenatal |
| Antibiotics | | range 0-3 | prescriptions for | |
| While | | | systemic | |
| Pregnant | | | antibiotics | |
| | | | during the | |
| | | | pregnancy | |
| Active | Continuous | Integer in | Number of | Prenatal |
| Atopy | | range 0-3 | atopic diagnoses | |
| While | | | during the | |
| Pregnant | | | pregnancy | |
| Infant | Dichotomous | Yes/No | Diagnosis of | Up to |
| Atopic | | | atopic dermatitis | 4 m |
| Dermatitis | | | before a food | |
| | | | allergy | |
| | | | diagnosis, but no | |
| | | | later than 4 | |
| | | | months of age | |
| Infant | Dichotomous | Yes/No | Systemics | Up to |
| Systemic | | | antibiotics | 4 m |
| Antibiotics | | | prescription | |
| | | | before 4 months | |
| | | | of age | |
| Infant | Dichotomous | Yes/No | Topical | Up to |
| Topical | | | antibiotics | 4 m |
| Antibiotics | | | prescription | |
| | | | before 4 months | |
| | | | of age | |

TABLE 12
Summary of ICD-9 Codes for Food Allergy Diagnosis

| ICD-9 Code | Explanation |
|---|---|
| V15.01 | Personal history of allergy to peanuts |
| V15.02 | Personal history of allergy to milk products |
| V15.03 | Personal history of allergy to eggs |
| V15.05 | Personal history of allergy to other foods |
| 995.3 | Allergy unspecified not elsewhere classified |
| 995.6 | Anaphylactic reaction due to unspecified food |
| 995.61 | Anaphylactic reaction due to peanuts |
| 995.63 | Anaphylactic reaction due to fruits and vegetables |
| 995.64 | Anaphylactic reaction due to tree nuts and seeds |
| 995.67 | Anaphylactic reaction due to milk products |

TABLE 13
Summary of Medications of Interest

| Medication | |
|---|---|
| Class | Generic Name |
| Systemic | Sulfadiazine, Amikacin, Amoxicillin, Clavulanic Acid- |
| Antibiotics | Amoxicillin, Azithromycin, |
| Prescribed | Benzathine benzylpenicillin, Ciprofloxacin/Dexamethasone, |
| During | Ciprofloxacin, Clarithromycin, Colistimethate sodium, |
| Pregnancy | Clindamycin, Sulfamethoxazole-Trimetoprime, Doxycycline, |
| | Benzylpenicillin, Erythromycin, Nitrofurantoin, Gentamicin, |
| | Methenamine hippurate, Levofloxacin, Cloxacillin, |
| | Metronidazole, Minocycline, Fosfomycin, Ofloxacin, |
| | Penicillin V, Ampicillin, Penicillin, Penicillin G (sodium, |
| | procaine), Piperacillin, |
| | Spiramycin, Roxithromycin, Tetracycline |
| Systemic | Clavulanic Acid + Amoxicillin, Benzatine benzylpenicilline, |
| Antibiotics | Sulfamethoxazole-Trimetoprime, |
| Prescribed | Erythromycine, Gentamicin, Amoxicillin, Penicillin V, |
| for Infants | Sulfamethoxazole-Trimetoprime, |
| | Erythromycin, Clarithromycin, Azithromycin, Amikacin |
| Topical | Gentamycin, Chloramphenicol, Fusidic Acid, Tetracycline, |
| Antibiotics | Mupirocin |
| Prescribed | |
| for Infants | |

While the present invention has been particularly described, persons skilled in the art will appreciate that many variations and modifications can be made. Therefore, the invention is not to be construed as restricted to the particularly described embodiments, and the scope and concept of the invention will be more readily understood by reference to the claims, which follow.

1. A method for preventing sensitization to a food allergen in a subject in need thereof, the method comprising administering a composition comprising an effective amount of said food allergen to any one of: (i) a mother being pregnant with said subject; (ii) a mother breastfeeding said subject, or both, thereby preventing sensitization to food allergen in the subject.

2. The method of claim 1, wherein said food allergen is selected from the group consisting of: peanut, sesame, milk, egg, shellfish, tree nut, fruit, fish, wheat, soy, and any combination thereof.

3. A method for preventing sensitization to peanut protein in a subject in need thereof, the method comprising administering a composition comprising an effective amount of peanut protein to any one of: to any one of: (i) a mother being pregnant with said subject; (ii) a mother breastfeeding said subject, or both, thereby preventing sensitization to peanut protein in the subject.

4. The method of claim 1, wherein said effective amount comprises a weekly dosage of 2.5 to 7.5 gr.

5. The method of claim 1, further comprising administering said composition to said mother during pregnancy.

6. The method of claim 1, wherein said composition is formulated for oral administration.

7. The method of claim 1, wherein said composition comprises said food allergen being encapsulated in a compound configured to inhibit or reduce degradation of said peanut protein in the gastrointestinal tract of said mother.

8. The method of claim 1, wherein said preventing comprises reducing the severity of sensitization to said food allergen in the subject.

9. The method of claim 1, wherein said preventing comprises reducing the titer of immunoglobulin E (IgE) targeting said food allergen in said subject, reducing wheal size developed by said subject in a skin prick test (SPT), or both.

10. The method of claim 9, wherein said reducing comprises reducing said titer of IgE by at least 5% compared to a control subject.

11. The method of claim 1, wherein said administering is multiple administering.

12. The method of claim 11, wherein said multiple administering comprises daily administering.

13. The method of claim 12, wherein said multiple administering comprises at least once a week administering.

14. The method of claim 1, wherein said composition is an edible composition comprising said food allergen.

15. The method of claim 1, wherein said subject is fed strictly on human breast milk.

16. The method of claim 1, further comprising a step preceding said administering, comprising selecting a subject at a risk of developing sensitization to said food allergen.

17. The method of claim 16, wherein said selecting is based on said subject having familial history of any one of: atopic dermatitis, food allergy, allergic rhinitis, asthma, eosinophilic esophagitis, or any combination thereof.

18. The method of claim 16, wherein said selecting is further based on: percent of siblings of said subject with an atopic condition, parent of said subject planning to exclusively breastfeed said subject, gender of said subject, season of birth of said subject, said subject being a firstborn, siblings of said subject with food allergy, systemic antibiotics of said mother while being pregnant with said subject, active atopy of said mother while being pregnant with said subject, infant atopic dermatitis in said subject, infant systemic antibiotics in said subject, infant topical antibiotics in said subject, and any combination thereof.

19. A method of generating a classifier suitable for predicting predisposition of a subject to a food allergen, by at least one processor, the method comprising:
a. receiving presence and/or level of a plurality of factors listed in Table 14 in a population of subjects afflicted with sensitization to said food allergen (‘sensitized population’) and in a population of subjects not afflicted with sensitization to said food allergen (non-sensitized population); and
b. at a training stage, training a machine learning model on a training set comprising:
(i) presence and/or level of said plurality of factors is subjects from said populations, and
(ii) labels associated with the sensitization or desensitization of a subject in said populations to said food allergen;
thereby generating a classifier suitable for predicting predisposition of a subject to said food allergen

20. The method of claim 19, further comprising at an inference stage, applying said classifier to a target set of plurality of factors associated with a target subject, thereby predicting predisposition of the subject to said food allergen.
